(19) United States
(12) Patent Application Publication
(10) Pub. No.: US 2019/0314417 A1
(43) Pub. Date: Oct. 17, 2019

(54) IMMUNOSUPPRESSIVE MESENCHYMAL CELLS AND METHODS FOR FORMING SAME

(71) Applicant: The Trustees of Columbia University In The City of New York, New York, NY (US)

(72) Inventors: Holly Wobma, New York, NY (US); Mariko Kanai, New York, NY (US); Gordana Vunjak-Novakovic, New York, NY (US)

(21) Appl. No.: 16/395,995

(22) Filed: Apr. 26, 2019

Related U.S. Application Data
(63) Continuation of application No. PCT/US2017/058686, filed on Oct. 27, 2017.
(60) Provisional application No. 62/530,617, filed on Jul. 10, 2017.
(60) Provisional application No. 62/413,696, filed on Oct. 27, 2016.

(51) Int. Cl. A61K 35/28; C12N 5/0775; C07K 14/725; A61K 35/17; A61P 37/00; A61P 29/00

(52) U.S. Cl. CPC A61K 35/28; C12N 5/0663; C12N 5/0668; C12N 5/0667; A61K 45/06; C07K 14/7051; A61K 35/17; A61P 37/00; A61P 29/00; C12N 5/0665

(57) ABSTRACT

The present disclosure describes immunosuppressive mesenchymal stromal cells and exosomes secreted from immunosuppressive mesenchymal stromal cells, and methods for their preparation. The disclosure also describes methods for treating subjects or preventing subjects at risk for conditions by administering the immunosuppressive mesenchymal stromal cells or secreted exosomes. The present disclosure also describes kits for preparing immunosuppressive mesenchymal stromal cells and exosomes secreted from immunosuppressive mesenchymal stromal cells.

### CROSS-REFERENCE TO RELATED APPLICATIONS

This application is a continuation of International Application No. PCT/US2017/058686 filed on Oct. 27, 2017, which claims the benefit of U.S. Provisional application No. 62/413,696 filed on Oct. 27, 2016, and claims the benefit of U.S. Provisional application No. 62/530,617 filed on Jul. 10, 2017, both of which are incorporated by reference herein.

### STATEMENT REGARDING FEDERALLY SPONSORED RESEARCH

This invention was made with government support under grant numbers UH3EB17103 and EB002520, awarded by the National Institute of Biomedical Imaging and Bioengineering. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 25, 2017, is named 16-50199-WO_SL.txt and is 7,568 bytes in size.

### BACKGROUND OF THE INVENTION

Mesenchymal stromal cells (MSCs) are studied for application in treating immune disorders due to their ability to promote immune suppression and tolerance, and their ability to be used allogeneically. Over the last decade, numerous clinical trials have evaluated MSCs for use in treating pathological immune responses in conditions such as inflammation, transplant rejection, and autoimmune disease (clinicaltrials.gov). These trials have been motivated by promising studies in vitro and in animal models, demonstrating that MSCs are hypo-immunogenic, can inhibit the development of an immune response, and skew diverse immune cell populations from pro-inflammatory towards anti-inflammatory/regulatory phenotypes. We hypothesized that inducing a stronger and more homogeneous immunosuppressive phenotype in MSCs prior to their administration would lead to better clinical outcomes and sought to identify an effective in vitro priming regimen.

While clinical trials have shown that MSCs are safe, they have also revealed that MSCs die within several days of administration yet still elicit a therapeutic effect. This has led to the “hit-and-run” hypothesis, which posits that the MSCs secrete paracrine factors during the first few days after injection, which cause immunomodulatory changes to the surrounding tissues that last longer than the MSCs themselves.

Even if persistent engraftment may not be necessary for MSCs to have an impact, there remains much room for improvement of cell therapies. For example, while clinical trials simply use culture-expanded MSCs, it is well established that MSCs are minimally immunosuppressive at baseline and adopt the immunosuppressive phenotype only after exposure to specific environmental cues. Subsequently, only a fraction of these naïve MSCs become immunosuppressive after injection, depending on an individual patient's internal cues. Assuming that only a fraction of cells are induced and that there is a delay in induction, the “hit” in the hit-in-run paradigm is not as effective as it would be if the cells started off by being homogeneously immunosuppressive at the time of injection. For this reason, in vitro priming regimens for inducing a more uniformly immunosuppressive MSC-phenotype prior to their administration are needed.

### SUMMARY

The present disclosure is directed to immunosuppressive mesenchymal stromal cells and methods for forming same.

In one embodiment, a method for preparing immunosuppressive mesenchymal stromal cells is provided. The method comprises the step of applying a pro-inflammatory cytokine to mesenchymal stromal cells in a hypoxic culture condition in vitro.

In another embodiment, a method for preparing immunosuppressive mesenchymal stromal cells is provided. The method comprises the steps of obtaining mesenchymal stromal cells isolated from a source and then applying a pro-inflammatory cytokine to the mesenchymal stromal cells in a hypoxic culture condition.

In another embodiment, a primed mesenchymal stromal cell is provided.

In another embodiment, a primed exosome is provided.

In another embodiment, a method for treating a subject experiencing a condition selected from the group consisting of cytokine storm, sepsis, autoimmune disease, transplant rejection, graft-vs-host disease, and inflammatory disease is provided. The method comprises the step of administering a primed mesenchymal stromal cell to the subject.

In another embodiment, a method for preventing a condition selected from the group consisting of cytokine storm, sepsis, autoimmune disease, transplant rejection, graft-vs-host disease, and inflammatory disease is provided. The method comprises the step of administering a primed mesenchymal stromal cell to a subject.

In another embodiment a method for treating a subject experiencing a condition selected from the group consisting of cytokine storm, sepsis, autoimmune disease, transplant rejection, graft-vs-host disease, and inflammatory disease is provided. The method comprises the step of administering a primed exosome to the subject.

In another embodiment a method for preventing a condition selected from the group consisting of cytokine storm, sepsis, autoimmune disease, transplant rejection, graft-vs-host disease, and inflammatory disease is provided. The method comprises the step of administering a primed exosome to a subject.

In another embodiment a method for screening the activity of an immunomodulatory agent is provided. The method comprises the steps of treating primed mesenchymal stromal cells with the immunomodulatory agent, isolating the primed mesenchymal stromal cells following said treatment with the immunomodulatory agent; and then subjecting the primed mesenchymal stromal cells to an immune activity assay to determine whether the immunomodulatory agent altered the immunosuppressive activity of the primed mesenchymal stromal cells.

In another embodiment, a composition comprising primed mesenchymal stromal cells in a pharmaceutically acceptable carrier is provided.

In another embodiment, a composition comprising primed exosomes in a pharmaceutically acceptable carrier is provided.

In another embodiment, a method for preparing immunosuppressive mesenchymal stromal cells is provided. The method comprises the steps of culturing mesenchymal stromal cells in the presence of primed exosomes and then isolating the cultured mesenchymal stromal cells.

In another embodiment, a kit for preparing immunosuppressive mesenchymal stromal cells comprising a first component and second component is provided. The first component comprises a pro-inflammatory cytokine and a hypoxia mimetic. The second component comprises frozen mesenchymal stromal cells.

In another embodiment, a kit for preparing immunosuppressive mesenchymal stromal cells comprising a first component, a second component, and a third component is provided. The first component comprises a pro-inflammatory cytokine. The second component comprises a hypoxia mimetic. The third component comprises frozen mesenchymal stromal cells

In another embodiment, a kit for preparing immunosuppressive mesenchymal stromal cells comprising a first component and a second component is provided. The first component comprises a pro-inflammatory cytokine. The second component comprises frozen mesenchymal stromal cells.

In another embodiment, a kit for use in preparing to administer immunosuppressive mesenchymal stromal cells is provided. The kit comprises a primed mesenchymal stromal cell.

In another embodiment, a kit for use in preparing to administer immunosuppressive therapy is provided. The kit comprises a primed exosome.

In any of the above embodiments, the mesenchymal stromal cells are exposed to the hypoxic culture condition for 1 hour to 48 hours.

In any of the above embodiments, the mesenchymal stromal cells are exposed to the hypoxic culture condition for 24 hours.

In any of the above embodiments, the mesenchymal stromal cells are exposed to the hypoxic culture condition for 48 hours.

In any of the above embodiments, the pro-inflammatory cytokine is selected from the group consisting of IL-1α, IL-1B, TNF-α, IFN-γ, IL-6, IL-12, IL-17, and IL-23.

In any of the above embodiments, the pro-inflammatory cytokine is IFN-γ.

In any of the above embodiments, IFN-γ is at a concentration of 0.1 ng/mL to 100 ng/mL.

In any of the above embodiments, IFN-γ is at a concentration of 1 ng/mL to 10 ng/mL.

In any of the above embodiments, the hypoxic culture condition comprises exposing the mesenchymal stromal cells to 37° C., 5% CO₂, and about 1% O₂ to about 5% O₂.

In any of the above embodiments, the hypoxic culture condition comprises exposing the mesenchymal stromal cells to 37° C., 5% CO₂, and 1% O₂.

In any of the above embodiments, the hypoxic culture condition comprises a hypoxia mimetic.

In any of the above embodiments, the hypoxia mimetic is selected from the group consisting of desferrioxamine, cobalt chloride, hydralazine, nickel chloride, diazoxide, and dimethyloxalyglycine.

In any of the above embodiments, the hypoxia mimetic is at a concentration of 50 μM to 200 μM.

In any of the above embodiments, further comprising the step of isolating exosomes secreted from the mesenchymal stromal cells following exposure to the pro-inflammatory cytokine and the hypoxic culture condition.

In any of the above embodiments, the source is selected from the group consisting of adipose tissue, umbilical cord, bone marrow, gingiva, and iPSCs.

In any of the above embodiments, further comprising administering an immunosuppressive agent to the subject.

In any of the above embodiments, the immunosuppressive agent is administered to the subject concurrently with the primed mesenchymal stromal cell.

In any of the above embodiments, the immunosuppressive agent is administered to the subject immediately prior to or after administering the primed MSC.

In any of the above embodiments, the immunosuppressive agent is selected from the group consisting of calcineurin inhibitors, steroids, microphenolate mofetil, anti-CD3 antibodies, aziothioprine, cyclophosphamide, ifosfamide, and other monoclonal antibodies used for immunosuppression.

In any of the above embodiments, further comprising an immunotherapy to the subject.

In any of the above embodiments, the immunotherapy is administered to the subject concurrently with the primed mesenchymal stromal cell.

In any of the above embodiments, the immunotherapy is administered to the subject immediately prior to or after administering the primed mesenchymal stromal cell.

In any of the above embodiments, the immunotherapy comprises chimeric antigen receptor T-cells.

In any of the above embodiments, the chimeric antigen receptor T-cells are administered to the subject concurrently with the primed mesenchymal stromal cell.

In any of the above embodiments, the chimeric antigen receptor T-cells are administered to the subject immediately prior to or after administering the primed mesenchymal stromal cell.

In any of the above embodiments, the immunosuppressive agent is administered to the subject concurrently with the primed exosome.

In any of the above embodiments, the immunosuppressive agent is administered to the subject immediately prior to or after administering the primed exosome.

In any of the above embodiments, the chimeric antigen receptor T-cells are administered to the subject concurrently with the primed exosome.

In any of the above embodiments, the chimeric antigen receptor T-cells are administered to the subject immediately prior to or after administering the primed exosome.

### BRIEF DESCRIPTION OF THE DRAWINGS AND FIGURES

The patent or application file contains at least one drawing executed in color. Copies of this patent or patent application publication with color drawing(s) will be provided by the office upon request and payment of the necessary fee.

FIG. 1A demonstrates the tri-lineage differentiation capacity of control adipose-derived MSCs.

FIG. 1B shows the expression of MSC surface markers and HLA-DR upon exposure to different priming conditions.

FIG. 2 shows testing of various ratios of control and dual primed MSCs at inhibiting either an MLR (top) or T-cells activated by CD2/CD3/CD28 beads (bottom).

FIG. 3 shows the induction of genes related to immunosuppression after 48 hours of priming.

FIG. 4A shows the kinetics of gene upregulation after MSC exposure to dual IFN-γ/hypoxia priming.

FIG. 4B shows the kinetics of gene expression after MSCs from three donors exposed to 48-hour dual IFN-γ/hypoxia priming, then returned to normoxia, and expression quanitifed after return to normal conditions.

FIG. 4C shows the kinetics of gene expression after MSCs exposed to 48 hours of dual IFN-γ/hypoxia priming (Day 2) compared with gene expression of MSCs exposed to a second round of stimulation after 7 days in normal condition (Day 11).

FIG. 5 shows mRNA transcriptional changes for MSCs stimulated via dual priming for two days vs four days, normalized to expression in control MSCs at the initial time point.

FIG. 6 shows protein expression in MSCs following 48 hours of priming.

FIG. 7A shows the immunosuppressive effects of differently primed MSCs in co-culture with mixed lymphocyte reactions (MLRs) normalized to the positive control (MLR without MSCs). The % of CD107+ cells in the entire CD8+ T-cell population is also shown, normalized to the positive control.

FIG. 7B shows the immunosuppressive effects of differently primed MSCs in co-culture with mixed lymphocyte reactions (MLRs), normalized to the % proliferated, CD25+, or CD107+ for the Control MSC co-culture (n=7-11).

FIG. 8 shows the immunomodulatory effect of differently primed MSCs in co-culture with MLRs.

FIG. 9 shows amounts of IFN-γ, TNF-α and IL-1α secreted into culture medium (by ELISA).

FIG. 10 shows the experimental design for evaluating the ability of primed MSCs to inhibit T-cells in mixed-lymphocyte reaction co-cultures (MSC-MLR).

FIG. 11A shows MSC-MLR co-culture experiments, depicting the variable inhibition by control and primed MSCs shown by % divided, % CD25+, % CD107+ in CD4 and CD8 T-cells (n=7-11).

FIG. 11B shows MSC-MLR co-culture experiments, depicting the GLUT1 expression in CD4+ and CD8+ T-cells at Day 1 and Day 3 of MSC-MLR co-cultures.

FIG. 11C shows MSC-MLR co-culture experiments, depicting the pro-inflammatory cytokine levels measured at Day 1 and 3 of MSC-MLR co-cultures.

FIG. 12 depicts the CD4+ T-cell memory panel characterization from MSC-MLR co-culture experiments.

FIG. 13 shows the relative soluble HGF levels in conditioned media from MSCs primed for 48 hours.

FIG. 14 shows protein expression after 48 hours of single or dual priming.

FIG. 15A shows the confirmation of disparate mRNA to protein level trends, depicting the relative IDO activity in MSCs after 48 hours of different priming regimens.

FIG. 15B shows the confirmation of disparate mRNA to protein level trends, depicting the relative HLA-G protein levels in MSC lysate after 48-hours of different priming regimens.

FIG. 16A depicts a Seahorse Mitochondrial Stress test data for MSCs cultured in priming conditions and then for 24 hours on Seahorse TC plates (10,000/well).

FIG. 16B shows glucose levels in MSC:MLR co-culture supernatant on Days 1 and 3 (standard average values are shown for duplicate readings).

FIG. 17A shows the influence of MSC priming on cell metabolism, where after 48 hours of priming, MSCs were replated at 10 000 cells per well into Seahorse Tissue Culture plates and evaluated by the Seahorse Mitostress kit. n=5

FIG. 17B shows the influence of MSC priming on cell metabolism, depicting GLUT1 expression in MSCs after 48 hours of priming.

FIG. 17C shows the influence of MSC priming on cell metabolism, depicting glucose and lactate levels in MSC-MLR co-culture experiments at Day 1 and Day 3. n=2.

FIG. 18A shows the effect of external L-lactic acid concentration on intracellular pH as revealed by increasing dye intensity with declining pH for n=3.

FIG. 18B shows the change in PBMC scatter properties as the external L-lactic acid concentration reaches 30 Mm.

FIG. 18C shows the effect of lactic acid concentration on T-cell division in response to Concanavalin A.

FIG. 19 shows the size distribution of exosomes in control, IFN-γ, hypoxia, and dual stimulation MSCs.

FIG. 20 shows dose-dependent incorporation of MSC-derived exosomes into activated PBMCs.

FIG. 21 represents a graphical abstract of the MSC single (IFN-γ or hypoxia) and dual (IFN-γ+hypoxia) in vitro priming regimens being evaluated for their capacity to promote a strong and homogenous immunosuppressive phenotype.

FIG. 22 shows IFN-γ titration, where MSCs were exposed to different concentrations of IFN-γ for 48 hours and then analyzed for IDO expression by flow cytometry.

FIG. 23 shows onset kinetics, where MSCs were exposed to 10 ng/mL IFN-γ and samples were taken at different time points to analyze expression of the immunosuppressive proteins IDO and PD-L1.

FIG. 24 shows hypoxia mimetic titration experiments where MSCs were exposed to either cobalt chloride (CoCl₂) or deferoxamine mesylate/desferrioxamine (DFO) at different concentrations and then analyzed for GLUT1, a marker for enhanced glycolysis.

### DETAILED DESCRIPTION

The present disclosure describes making an immunosuppressive MSC phenotype that is more likely to be successful as a cell therapy for multiple disorders that involve a pathological immune response (inflammation, autoimmune disease, graft rejection). Current MSC therapies use unprimed MSCs, which are not immunosuppressive at baseline, and the MSCs also die shortly after injection. We sought to overcome these challenges using a biologically inspired strategy to prime our MSCs prior to injection, namely hypoxia and pro-inflammatory cytokines.

Our hypothesis of the benefits of combining inflammation and hypoxia is supported by our data. IFN-γ and hypoxia were found to upregulate distinct immunosuppressive proteins at the mRNA and, when combined, synergistically improved expression of multiple immunosuppressive proteins at the protein level. Importantly, while priming MSCs with either of these cues, alone, led to a more immunosuppressive MSC phenotype than unprimed MSCs, combining IFN-γ and hypoxia led to a much more suppressive phenotype. We believe this is related to the enhanced expression of immunosuppressive proteins that occurs when these two cues are combined and because of the metabolic effect hypoxia has on MSCs. We have shown that hypoxia priming causes MSCs to be more dependent on glycolysis, greatly increasing their glucose consumption and lactate production. Since inflammatory/activated T-cells and macrophages also depend on glucose and glycolysis, our MSCs may be outcompeting them for nutrients, which is a scenario that has been described as a mechanism for immune escape in tumors. Similarly, inhibition of inflammatory cells by high lactate levels is another means of immune escape. Lastly, the switch from oxidative phosphorylation to glycolysis by the MSCs that see hypoxia (either alone or in combination with IFN-γ) means they are less oxygen dependent, which is consistent with accounts of better survival of hypoxia-primed MSCs in animal models of ischemic damage (where oxygen is limited).

In summary, we use interferons/pro-inflammatory cytokines (not TLR3 ligands) and hypoxia to induce an anti-inflammatory, pro-survival MSC phenotype, which is opposite to how interferons are framed in the prior art and goes beyond the description of the role of hypoxia as simply a factor that enables “stemness”.

As described above, in one aspect, a cell culture regimen for enhancing the potential of mesenchymal stem cells/stromal cells (MSCs) for use as cell therapies in treating or preventing disorders of unwanted immune response (e.g. autoimmune disease, inflammation, graft rejection) is provided.

Administration of primed MSCs or primed exosomes to humans can be via local or systemic administration of the MSCs or exosomes suspended in buffer, basal media, or other formulation. Local administration could include, but is not limited to, administration at wound sites like diabetic ulcers or burns, intra-muscular injection, spinal cord injection, administration of a cardiac patch on the heart, or injection into the superior vena cava, mesenteric blood vessels, or coronary artery. Systemic administration could include, but is not limited to, IV injection, intra-arterial injection, or intraperitoneal injection. The dose of MSCs or exosomes and timing of administration will be optimized using routine methods. For a general discussion of using MSCs as a cell-based therapy in humans, see Jun Zhang et al., The Challenges and Promises of Allogeneic Mesenchymal Stem Cells for Use as a Cell-based Therapy, STEM CELL RESEARCH & THERAPY, Dec. 1, 2015, which is incorporated herein by reference in its entirety.

MSCs could be prepared for clinicians in a kit. Kits could potentially include pre-primed MSCs or exosomes. Alternatively, a kit could contain frozen MSCs in conjunction with materials that the physician/hospital could use to prime the MSCs themselves. The materials could include a combination or mixture of pro-inflammatory cytokines and/or hypoxia mimicking agent.

The MSCs of the present disclosure can be derived from adipose tissue, umbilical cord, bone marrow, gingiva, iPSCs, or any other source known in the art for deriving MSCs. Cells similar to MSCs such as multipotent adult progenitor cells may also be primed according to the present disclosure.

It should be understood that hypoxic culture conditions can be created a variety of ways. Hypoxic culture conditions could be created by lowering the oxygen in the culture environment (such as culturing in 1%-5% O₂). Another way to create hypoxic culture conditions would be to add a hypoxia mimicking agents to the culture environment, such as desferrioxamine/deferoxamine mesylate, cobalt chloride, hydralazine, nickel chloride, diazoxide, or dimethyloxalyglycine. A hypoxic culture condition could also be created by application of factors, such as hypoxia-inducing factor (HIF), to the culture media, triggering cellular responses similar to that of environmental hypoxia.

As used herein, the term “primed mesenchymal stromal cell” is defined as a mesenchymal stromal cell exposed in vitro to a pro-inflammatory cytokine and hypoxic culture conditions.

As used herein, the term “primed exosome” is an exosome from a primed mesenchymal stromal cell.

As used herein, the term “hypoxia mimetic” is any formulation that stabilizes hypoxia inducible factor or induces a related hypoxic response.

### EXAMPLES

The present invention is demonstrated in the following examples, it being understood that these are for illustrative purposes only, and the invention is not intended to be limited thereto.

### Materials and Methods

For all examples herein, the following materials and methods were used:

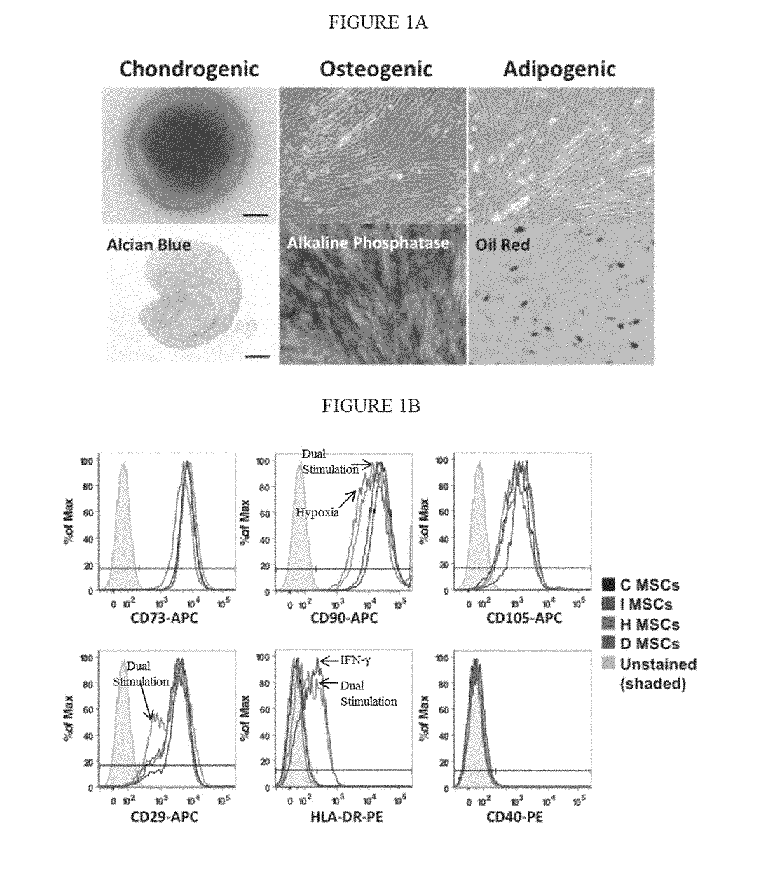

MSC Culture and Priming. Frozen vials of MSCs from fully de-identified human lipoaspirates were kindly provided by Dr. Jeffrey Gimble (Tulane University) and tested for successful tri-lineage differentiation as well as positive surface expression of in vitro MSC markers and negative expression of antigen-presenting cell markers (FIGS. 1A & 1B). MSCs from 3 different donors were used in experiments to demonstrate the generalizability of the cell responses to priming regimens. Cells were cultured in MSC media (DMEM 11965 with 10% FBS and 1% Pen/Strep) and plated into 6-well plates for priming experiments at passage 5. FIG. 1A depicts the tri-lineage differentiation capacity of control adipose-derived MSCs as demonstrated by histological staining for chondrocytes (Alcian blue), osteoblasts (Alkaline Phosphatase), and adipose cells (Oil Red). FIG. 1B shows relative surface protein expression for the MSC markers: CD29, CD73, CD90, and CD 105, and antigen presenting cell markers: HLA-DR and CD40, after 48 hours of priming. The only antigen presenting cell marker present was HLA-DR, which was seen in MSCs that experienced IFN-γ in their priming regimen (39.2%+from IFN-γ priming; 30.2%+from dual priming).

MSCs were grown to confluence in 6-well plates and subsequently exposed to: control conditions (normoxia, regular MSC media), individual IFN-γ or hypoxia priming, or dual IFN-γ/hypoxia priming (4 different conditions). IFN-γ (Peprotech) was used at a concentration of 100 ng/mL. A hypoxic culturing environment was achieved using a New Brunswick Galaxy 145 incubator at 37° C., 5% CO₂, and 1% O₂. Priming was applied for 48 hours unless otherwise noted, and the MSCs were then analyzed for gene and protein expression or evaluated in functional studies. Collected MSCs always had a viability of >95%, and there were no differences in viability between priming groups.

qRT-PCR. A comprehensive list of 15 genes implicated in MSC-based immunosuppression/protection was established based on published studies. MSC priming experiments were repeated 4 times using MSCs from 3 different donors.

RNA was isolated using the RNAqueous Micro Kit (Life Technologies) and quantified using a Nanodrop ND1000. RNA was treated with DNase I, Amplification Grade Kit (Invitrogen) and converted to cDNA using the High Capacity cDNA Reverse Transcription Kit (Applied Biosystems) following the manufacturer's protocol.

Quantitative RT-PCR (qRT-PCR) analysis was performed using 20 ng cDNA per reaction, and the SYBR Green PCR Master Mix (Applied Biosystems). The expression of target genes at each time point was normalized to GAPDH and subsequently to the unprimed phenotype at its baseline time point (2^{-ΔΔCt}). All primers (TABLE 1) were checked for theoretical target gene specificity using NCBI Primer-BLAST.

TABLE 1
Forward and reverse primers
for immunomodulatory genes.

| | Forward | Reverse |
|---|---|---|
| GAPDH | AAGGTGAAGGTCGGA | GGGGTCATTGATGGCAACAATA |
| | GTCAAC | (SEQ ID NO: 17) |
| | (SEQ ID NO: 1) | |
| HLA-G | GAAGAGGAGACACGG | TGGCCTCATAGTCAAAGACA |
| | AACA | (SEQ ID NO: 18) |
| | (SEQ ID NO: 2) | |
| HLA-E | ATGGAACCCTCCTTT | GGCTCCAGGTGAAGCAGC |
| | TACTC | (SEQ ID NO: 19) |
| | (SEQ ID NO: 3) | |
| HGF | GGTGACCAAACTCCT | ACCTCTGGATTGCTTGTGAAA |
| | GCCA | (SEQ ID NO: 20) |
| | (SEQ ID NO: 4) | |
| COX2 | CAGCCATACAGCAAA | ATCCTGTCCGGGTACAAT |
| | TCC | (SEQ ID NO: 21) |
| | (SEQ ID NO: 5) | |
| iNOS | TCATCCGCTATGCTG | CCCGAAACCACTCGTATTTGG |
| | GCTAC | (SEQ ID NO: 22) |
| | (SEQ ID NO: 6) | |
| IDO | TCTCATTTCGTGATG | GTGTCCCGTTCTTGCATTTGC |
| | GAGACTGC | (SEQ ID NO: 23) |
| | (SEQ ID NO: 7) | |
| LIF | CCAACGTGACGGACT | TACACGACTATGCGGTACAGC |
| | TCCC | (SEQ ID NO: 24) |
| | (SEQ ID NO: 8) | |
| IL-10 | TCAAGGCGCATGTGA | GATGTCAAACTCACTCATGGCT |
| | ACTCC | (SEQ ID NO: 25) |
| | (SEQ ID NO: 9) | |
| TGF-β | GGCCAGATCCTGTCC | GTGGGTTTCCACCATTAGCAC |
| | AAGC | (SEQ ID NO: 26) |
| | (SEQ ID NO: 10) | |
| TSG-6 | GGGCAGAGTTGGATA | TGCGTGTGGGTTGTAGCAATA |
| | CCCC | (SEQ ID NO: 27) |
| | (SEQ ID NO: 11) | |
| CD59 | TTTTGATGCGTGTCT | ATTTTCCCTCAAGCGGGTTGT |
| | CATTACCA | (SEQ ID NO: 28) |
| | (SEQ ID NO: 12) | |
| PD-L1 | GGACAAGCAGTGACC | CCCAGAATTACCAAGTGAGTCCT |
| | ATCAAG | (SEQ ID NO: 29) |
| | (SEQ ID NO: 13) | |
| Arginase-1 | GTGGAAACTTGCATG | AATCCTGGCACATCGGGAATC |
| | GACAAC | (SEQ ID NO: 30) |
| | (SEQ ID NO: 14) | |
| Galectin-1 | TCGCCAGCAACCTGA | GCACGAAGCTCTTAGCGTCA |
| | ATCTC | (SEQ ID NO: 31) |
| | (SEQ ID NO: 15) | |
| Galectin-3 | GTGAAGCCCAATGCA | AGCGTGGGTTAAAGTGGAAGG |
| | AACAGA | (SEQ ID NO: 32) |
| | (SEQ ID NO: 16) | |

For kinetics studies, a subset of genes was evaluated (HLA-G, IDO, PD-L1, and COX-2). To study the onset kinetics of gene upregulation, MSCs were stimulated by dual priming (or kept under control conditions) for 48 hours, with RNA samples collected at 0, 4, 12, 24 and 48 hours. For offset kinetics studies, the same 48-hour regimen was followed, but the cells were subsequently returned to normoxia in fresh MSC media. Samples were taken both at 4 days and 7 days after return to control conditions, with a standard media change halfway through the study. The experiment was repeated after the second 48-hour stimulation to evaluate if gene expression changes could be reinduced.

MSC Protein Expression Studies. Immediately following priming, MSCs were analyzed for intracellular and surface markers using a BD FACS CANTOII flow cytometer (always >20,000 event counts). For intracellular proteins, cells were fixed and permeabilized using the BD Cytofix/Cytoperm kit. Cells were stained in BD BSA Stain Buffer for 20 minutes at 4° C. and then washed twice with stain Buffer. A complete list of antibody clones and dilutions can be found in TABLE 2 and TABLE 3.

TABLE 2
Flow cytometry antibody details for both MSC and PBMC
staining. CF/CP denotes initial treatment with BD Cytofix/Cytoperm.
Staining volume = 100 kL throughout.

| | | | Test | Staining |
|---|---|---|---|---|
| Target | Clone | Fluorophore | Size | Type |
| CD3 | UCHT1 | PerCP-CyTMS.5 | 1:20 | Surface |
| CD4 | RPA-T4 | PE-Cy7 | 1:80 | Surface |
| CD8 | SK1 | APC-Cy7 | 1:40 | Surface |
| CD107a | H4A3 | PE | 1:5 | Surface |
| CD25 | M-A251 | APC | 1:5 | Surface |
| CD95 | DX2 | PE | 1:20 | Surface |
| CD45RA | HI100 | V500 | 1:20 | Surface |
| CCR7 | 150503 | FITC | 1:20 | Surface |
| HLA-G | MEM-G/9 | FITC | 1:10 | Fixed-CF/CP |
| IDO | eyedio | eFluor660 | 1:20 | Fixed-CF/CP |
| CD274 (PD-L1) | MIH1 | eFluor450 | 1:20 | Surface |
| COX-2 | AS66 | FITC | 1:20 | Fixed-CF/CP |

TABLE 3
Flow cytometry antibody details for MSC and PBMC staining.

| | | | Antibody | |
|---|---|---|---|---|
| Target | Clone | Fluorophore | Dilution | Source |
| CD3 | UCHT1 | PerCP- | 1:20 | BD |
| | | CyrM5.5 | | |
| CD4 | RPA-T4 | PE-Cy7 | 1:80 | BD |
| CD8 | SK1 | APC-Cy7 | 1:40 | BD |
| CD107a | H4A3 | PE | 1:5 | BD |
| CD25 | M-A251 | APC | 1:5 | BD |
| CD45RA | HI100 | V500 | 1:20 | BD |
| CCR7 | 150503 | FITC | 1:20 | BD |
| HLA-G | MEM-G/9 | FITC | 1:10 | Abcam |
| IDO | eyedio | eFluor660 | 1:20 | eBioscience |
| CD274 (PD-L1) | MIH1 | eFluor450 | 1:20 | eBioscience |
| COX-2 | AS66 | FITC | 1:20 | Cayman |
| HLA-E | 3D12 | APC | 1:10 | Miltenyi |
| GLUT1 | SPM498 | APC | 1:500 | Abcam |

Mixed Lymphocyte Reactions (MLRs). For MLRs, a peripheral blood mononuclear cell (PBMC) bank of cryopreserved cells was made using fully de-identified samples from 10 different donors, to generate a set of stimulator-responder pairs. PBMCs were isolated from fresh leukopaks (New York Blood Center) using Histopaque-1077 (Sigma)-based density gradient centrifugation, washed twice with bone marrow medium (BMM; Media 199 with 1% HEPES, 1% Pen/Strep, and 20 kU DNAse I), treated with ACK lysis Buffer (Thermo Fisher) for red cell lysis, and cryopreserved.

MSCs were collected after 48-hour priming using 0.25% trypsin-EDTA, and seeded at either 1×10⁶/mL or 2×10⁶/mL in 40 μL (i.e. 40,000 or 80,000 cells total) in 96-well U-bottom plates in complete AIM-V supplemented with 5% heat-inactivated human AB serum, 1% Pen/Strep, 1% HEPES, and 50 μM 2-mercaptoethanol (cAIM-V).

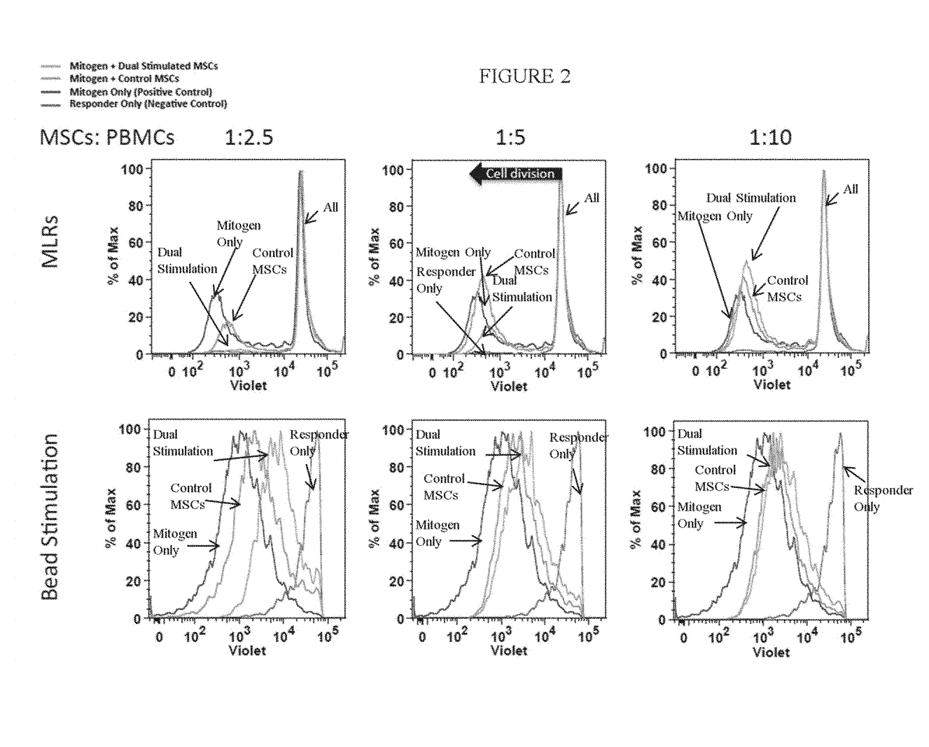

Two batches of allogeneic PBMCs were thawed in 1:1 BMM:cAIM-V and washed twice. Responder PBMCs were stained with BD Violet Proliferation Dye per manufacturer's instructions (final concentration: 1 μM). Stimulator PBMCs were inactivated using 30 Gy X-ray irradiation (X-RAD 320) or 10 mg/mL Mitomycin C (always provided similar results in comparison studies). Stimulator and responder PBMC cell concentrations were adjusted to 2.5×106/mL, and 80 kiL of each cell Suspension (i.e. 200,000 cells) was layered on top of previously plated MSCs. Thus, the stimulator to responder ratio was 1:1 and the MSC to responder PBMC ratio was 1:2.5 or 1:5, which were the ratios identified in pilot studies as optimal for comparisons (FIG. 2).

MLR experiments were run for 5 days, with 50 μL of cAIM-V added halfway through. For end-point analysis, two antibody panels were used: CD3/4/8/25/107a (activation/cytotoxicity) and CD3/4/8/45RA/197 (naive vs. memory). Primary conjugated antibodies (BD Pharmingen) were used at the recommended test size, save for CD4 and CD8, which were used at 1:80 and 1:40 dilutions, respectively (again, see TABLE 2 and TABLE 3 for clones). All surface staining was done without fixation in BD BSA Stain Buffer. Flow cytometry analysis was done within 30 minutes of staining. When analyzing CD4+ and CD8+ subpopulations, each group was normalized to that of the MLR with no MSCs (positive control set to 100%) by dividing each experiment's % divided (violet negative), % CD25+, or % CD107+ by the corresponding values for the MLR only condition. These group data were then averaged over 7-11 experiments.

For some experiments, extra MLR-MSC reactions were set up for various Day 1 and Day 3 analyses. Pro-inflammatory cytokine levels were detected using a Human Cytokine 16-Plea ELISA kit (PBL Assay Science, Piscataway, N.J.). Supernatant glucose levels were determined by the Hormone and Metabolite Core Laboratory at Columbia University Medical Center. Supernatant lactate levels were determined by the colorimetric L-Lactate Assay Kit (Abcam, Cambridge, Mass.), following an initial deproteinization step (as instructed) and dilution 1:3 to be within the range of the kit. Cells from Day 1 and Day 3 MSC-MLR experiments were also collected, fixed, permeabilized, and stained for the GLUT1 transporter.

Metabolic Assays (Seahorse). Following priming, MSCs from the different priming regimens were plated on Seahorse tissue culture plates (10,000 cells in 100 μL) and incubated overnight at 37° C. in a standard 5% CO₂ incubator. The following day, wells were washed twice with Seahorse Assay medium (100 mL) containing (1 ml of 200 mM L-glutamine, 1 ml of 100 mM Sodium Pyruvate, and 400 μL of 45% D-Glucose; pH 7.4) and the plate was kept in a non-CO₂ incubator for 2 hours prior to running the Mito Stress Assay (Oligomycin 1 μM, FCCP 1 μM, Rotenone/Antimycin 1 μM). Data were analyzed using the Wave software.

Flow Cytometry for MSC Proteins. MSCs were initially stained for standard MSC markers (CD29, CD73, CD90, CD105) and those that might suggest antigen presenting capacity (CD40, HLA-DR), both before and after priming. MSCs were primed for 24-48 hours. They were then stained for immunomodulatory proteins that had shown up-regulation at the mRNA level from qRT-PCR (HLA-G, COX-2, IDO, PD-L1, HLA-E; also GLUT1 for metabolic studies). For intracellular proteins, cells were fixed, permeabilized and stained using the reagents in the BD Cytofix/Cytoperm kit. For surface staining, cells were stained in BD BSA Stain Buffer for 20 minutes at 4° C. and then washed twice with stain buffer. All data collection was performed on a BD FACS CANTOII flow cytometer followed by analysis in FlowJo (Ashland, Oreg.). A complete list of antibody clones and dilutions can be found in TABLE 3.

IDO activity assay. MSCs that had just been primed for 48 hours were re-plated in fresh control ASC media at 10 000 cells per well in 96-well plates and left overnight to attach. Measurement of IDO activity was achieved via a kit (BPS Bioscience, San Diego, Calif.) as per the manufacturer's protocol, with the modification that no transfection of IDO was performed, and it was simply measured in the MSCs of different priming conditions. Samples were performed in replicates of 6.

HLA-G Western blot. After washing 2× with PBS, primed MSCs were lysed on ice for 30 minutes with NP-40 Lysis buffer containing phosphatase and protease inhibitors each at a 1:50 ratio (Thermo Fisher). Lysates were centrifuged at 4° C., 13,600×g for 15 minutes. Protein concentrations were determined via a BCA protein assay (Thermo Fisher). After the protein concentrations from different MSC priming groups were normalized, the supernatant was diluted with 1:1 with 2× Laemmli Sample Buffer (Bio-Rad, Hercules, Calif.) and boiled at 95° C. for 5 minutes. Protein samples were resolved by SDS-PAGE in a 4-20% precast polyacrylamide gel (Bio-Rad) and electrotransferred onto a polyvinylidene difluoride (PVDF) membrane. After transfer, the membrane was blocked with 5% bovine serum albumin (BSA) TBST for 1 hour and then probed with an anti-HLA-G primary antibody (1:500, OriGene Technologies) at 4° C. overnight. The membrane was then washed with TBST 3× and exposed to a goat anti-rabbit IgG AlexaFluor 680 secondary antibody (1:10 000, Thermo Fisher) for 1 hour. After three final washes, the membrane was imaged on a Licor Odyssey scanner (Lincoln, Nebr.).

HGF ELISA. MSC supernatant collected at the end of 48 hour priming was collected, spun down to pellet cell debris, and then transferred to fresh tubes for storage at −80° C. ELISA samples were brought to room temperature and used undiluted with the Invitrogen Human HGF ELISA kit (Carlsbad, Calif.), as per manufacturer's instructions.

Lactic acid titration. PBMCs were thawed, as previously described, and stained in HEPES buffered saline at 37° C. for 30 minutes with pHrodo® Red AM (Thermo Fisher) as per manufacturer's instructions (1000× dilution of dye). After a single wash with HEPES buffer, samples were divided into 5 Eppendorf tubes, spun down, and resuspended in cAIM-V with L-lactic acid (Sigma) added to make concentrations of: 0 mM, 5 mM, 10 mM, 20 mM, and 30 mM. Cells were resuspended in these new media and incubated at 37° C. for 30 minutes. Immediately prior to each flow cytometry reading, each sample was diluted 1:9 with BD Stain Buffer and mixed well. This neutralized external pH and diluted media components without permitting time for intracellular lactate to be exported. For lymphocyte proliferation experiments, PBMCs were thawed and stained with BD Violet Proliferation dye, as described previously, and resuspended in cAIM-V with L-lactic acid added to the concentrations above. Cells were plated in 96-well U-bottom plates at 200 000 cells per well. ConA was added to be 5 μg/mL, and PBMCs were analyzed by flow cytometry on Day 3.

Mass spectrometry. MSC plates were washed×3 with ice-cold PBS to remove residual FBS and added cytokines. Lysis buffer consisting of TBS with 3% SDS and 50 μL protease inhibitors (Sigma) was added to each MSC plate. The lysate was collected and proteins were precipitated in chloroform/methanol. Mass spectrometry was performed at the Quantitative Proteomics and Metabolomics Center at Columbia University with an UltiMate 3000 RSLCNano ultrapressure liquid chromatograph coupled to a Q Exactive HF (Orbitrap) mass spectrometer (Thermo Fisher, Bremen, Germany).

Statistical Analysis. One-way ANOVA analysis in conjunction with Tukey post-hoc tests was used to compare the PCR, MLR, flow cytometry, ELISA, lactate and glucose results from different priming groups using GraphPad Prism 6 software. Data were presented as mean±standard deviation; p<0.05 was considered statistically significant.

### Example 1

Here we propose a specific in vitro priming protocol of MSCs that can enhance their immunosuppressive qualities. We first compared combinations of three different stimuli: hypoxia (1% O₂), IFN-γ (100 ng/mL), and IL-10 (10 ng/mL). Based on gene expression studies for >12 immunosuppressive/protective factors, the conditioning regimen of the combination of IFN-γ and hypoxia for two days led to the target expression of genes known to induce immunosuppression. Those with the greatest fold increase post-stimulation included PDL1, IDO, HLA-G, and COX2, the first two being responsive to the IFN-γ component, while the latter two were mainly upregulated by hypoxia. Importantly, the expression of these genes remained increased over baseline for over one-week post-stimulation, a phenomenon that could be re-capitulated upon re-stimulation.

Here, we explored combined IFN-γ and hypoxia priming of human MSCs. After 48 h of stimulation, several immunosuppressive factors were upregulated, most dramatically IDO and PDL1 (by IFN-γ) and COX2 and HLA-G (by hypoxia). Gene expression changes persisted for one-week post stimulus removal. While IFN-γ stimulation upregulated expression of classical MHC proteins, allogeneic reaction with human PBMCs was not observed. In fact, MSCs preconditioned with IFN-γ/hypoxia suppressed expansion of both CD4+ and CD8+ T cell populations in response to CD2/CD3/CD28 bead stimulation to a greater extent than unconditioned MSCs. These in vitro data suggest use of uniformly immunosuppressive populations of MSCs in cell therapies. To investigate whether there is also a greater potential for cell homing following combined stimulation, we studied changes in chemokine receptor expression, and observed a consistently greater expression of CCR1 and CCR7 relative to other chemokine receptors.

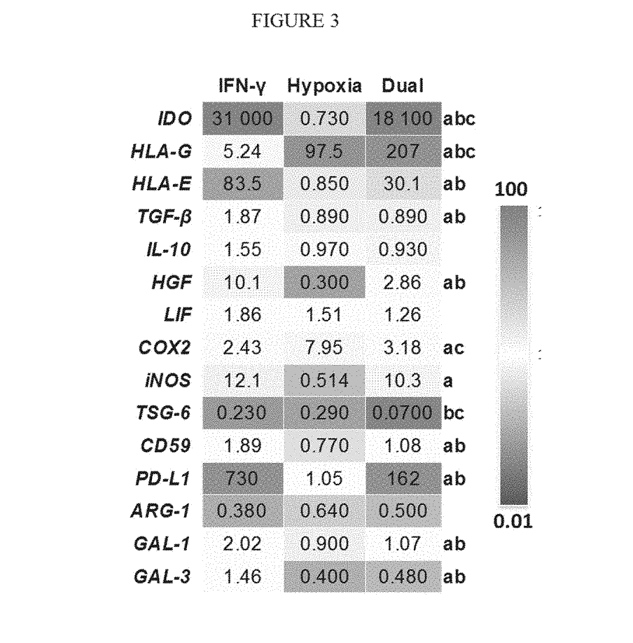

Dual priming leads to multiple immunomodulatory factors being upregulated with distinct contributions from IFN-γ and hypoxia. Two-day priming by either IFN-γ or hypoxia resulted in upregulation of distinct immunomodulatory genes as evidenced by qRT-PCR (FIG. 3). FIG. 3 represents the mRNA data obtained by qRT-PCR after 48 hours of priming by IFN-γ, hypoxia, or dual IFN-γ/hypoxia in a representative experiment. Data in FIG. 3 are normalized to the expression in control MSCs (normoxia and regular MSC media, n=4-6). The bar scales the fold difference from 0.01 to 100 (and saturates at either end). Significant differences between groups, as determined by ANOVA and Tukey post-hoc tests (p<0.05), are shown at the right of each gene as indicated: a, IFN-γ vs. hypoxia stimulation; b, IFN-γ vs. dual stimulation; and c, hypoxia vs. dual stimulation. The genes induced by IFN-γ included: HGF, iNOS, HLA-E and, most significantly, PD-L1 and IDO, the expression of which increased by 102 fold and 104, respectively, when compared to MSCs under control conditions. IFN-γ priming led to >5-fold induction of HLA-G, HLA-E, HGF, iNOS, and, most notably, PD-L1 and IDO that were induced by 730-fold and 31,000-fold, respectively. In contrast, hypoxia priming led to greater induction of HLA-G than IFN-γ priming (100 fold vs. 5 fold) and induction of COX-2. While there was some mild induction of COX-2 and HLA-G by IFN-γ, these genes were more strongly induced by hypoxia. All factors upregulated by IFN-γ and hypoxia individually, were induced when these two priming stimuli were combined, such that 7/15 factors were significantly upregulated. While the expression of IDO and PD-L1 was not as high from dual priming as single priming alone, the level of induction was still the same order of magnitude. By contrast, HLA-G was more induced by dual priming than by single priming with either stimulus alone. Overall, the combination of the two priming conditions resulted in an augmentation of the two transcriptional upregulation patterns obtained by IFN-γ and hypoxia individually (FIG. 3). The effects were not exactly additive, as the expression of some genes (e.g. IDO, HLA-E, PD-L1) induced by IFN-γ was partially subdued by dual priming, while the induction of others (e.g., HLA-G) was instead potentiated.

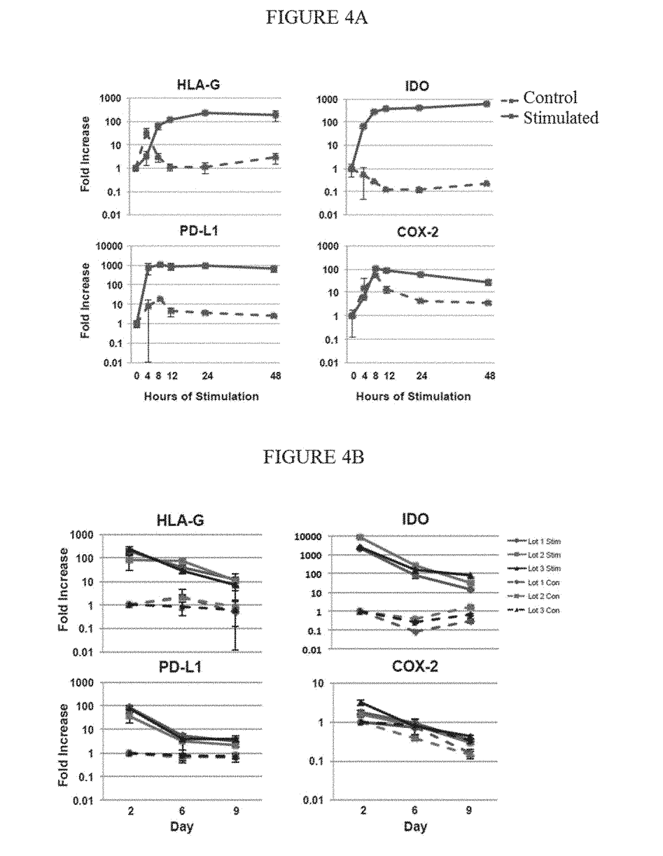

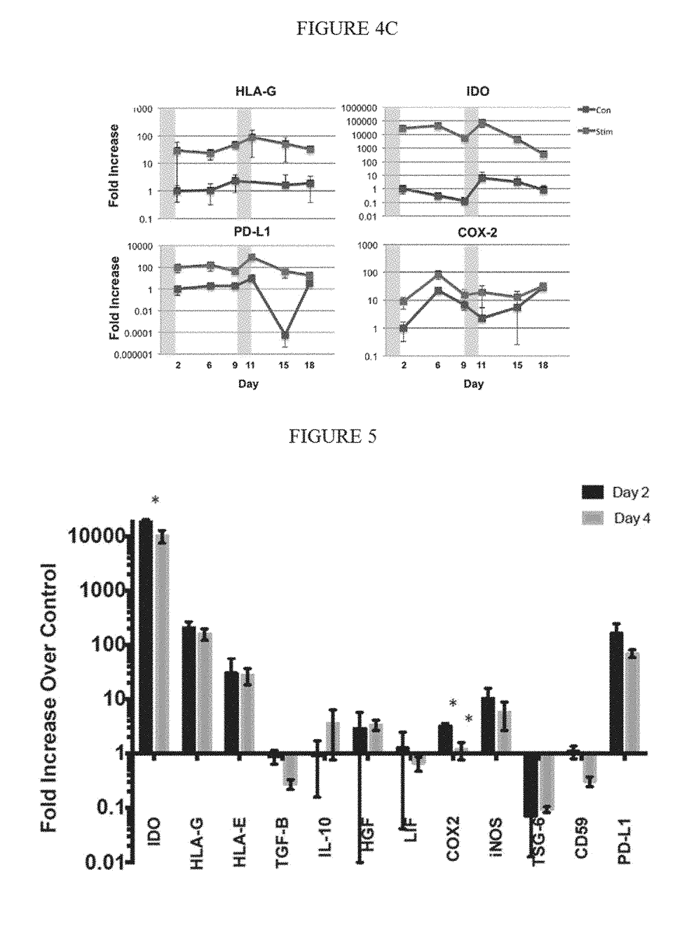

Most highly induced genes are at peak expression at 48 hours. To determine the kinetics of gene induction, a subset of genes was followed over 48 hours for control MSCs and dual primed MSCs: two genes highly induced by IFN-γ (IDO, PD-L1) and two genes induced by hypoxia (HLA-G, COX-2). qRT-PCR was performed an samples taken over multiple time points over the first 48 hours of priming. Induction of gene expression was already evident by 4 hours, although it generally took 8-12 hours to reach peak levels of mRNA (FIG. 4A). The peak expression was maintained across the 48-hour period for HLA-G and PD-L1, whereas IDO continued to trend upwards, and COX-2 underwent a transient expression. Unexpectedly, COX-2 had over tenfold higher mRNA expression when sampled at 8 hours than 48 hours, revealing a much greater induction than was initially inferred from the 48 hour priming experiments. However, since the other genes followed were at their peak at 48 hours, and there is a delay in protein translation over mRNA transcription, a 48-hour duration was maintained for all MSC priming experiments. Priming for longer than 48-hours was not shown to be beneficial (FIG. 5).

### Example 2

Genes induced by dual priming stay upregulated for up to one week and can be re-induced. To better understand how gene expression changes would persist in the setting of therapeutic application, dual primed MSCs were returned to control conditions, and qRT-PCR for HLA-G, IDO, PD-L1, and COX-2 was performed after 4 days and 7 days. Notably and unexpectedly, for all three MSC donors, HLA-G, IDO, and PD-L1 remained significantly upregulated after being returned to control conditions for 7 days, although a noticeable drop from their peak expression could be seen by day 4 (FIG. 4B). Consistent with the kinetic studies that showed a decline in COX-2 expression by 48 hours, COX-2 expression continued to mildly decline for MSCs that had previously been kept in either control or priming conditions. Since primed MSCs may be re-exposed to inflammatory and hypoxic cues in the patient, the priming regimen was repeated after being returned to control conditions for seven days, and the induction after round 1 (Day 2) and round 2 (Day 11) of 48 hour priming was compared. All four genes could be re-induced, and mRNA levels for IDO and PD-L1 were significantly higher upon re-exposure to the same priming cues (FIG. 4C).

### Example 3

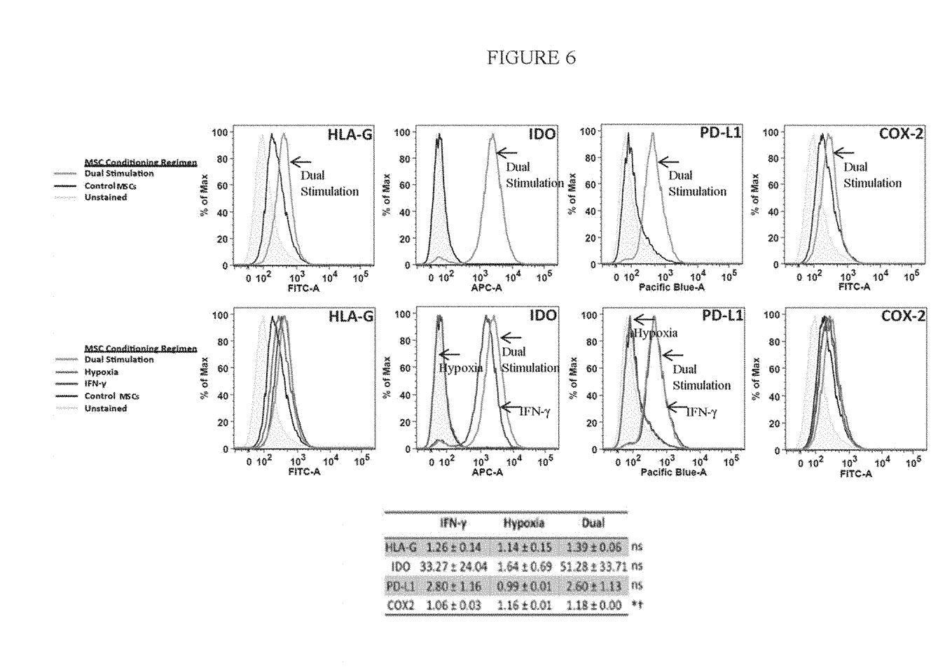

Dual priming induces immunomodulatory factors at the protein level. In FIG. 6, data are shown for various 48-hour priming regimens. Histograms are from a representative experiment. For clarity, only control MSCs vs. dual primed MSCs are shown on the left, whereas all conditions are shown on the right. The table at the bottom of FIG. 6 shows the mean fluorescence intensity (MFI) of the primed MSCs normalized to the MFI of Control MSCs for n=3 experiments. Significance is shown as (*) for IFN-γ vs. hypoxia stimulation and (t) for IFN-γ vs. dual stimulation. Flow cytometry for HLA-G, IDO, PD-L1, and COX-2 confirmed that they were upregulated at the protein level after dual priming for 48 hours (FIG. 6, top). Considering both single factor and dual factor priming regimens, there were some different patterns at the protein level as compared with the initial PCR findings (FIG. 6, bottom). At the protein level, IDO had slightly more induction by dual priming (although nonsignificant) than by IFN-γ alone, which differs from PCR findings that showed a reduction in mRNA expression in the setting of dual priming. HLA-G, which was more greatly induced by hypoxia at the mRNA level, was more strongly induced by IFN-γ than hypoxia at the protein level. Consistent with PCR findings, PD-L1 was upregulated more strongly by IFN-γ and dual priming, while COX-2 was induced more strongly by hypoxia. Of note, the difference in protein levels of COX-2 between the hypoxia/dual-primed MSCs and IFN-γ primed MSCs was modest but statistically significant. The protein expression for the four genes was similar for IFN-γ primed MSCs and dual primed MSCs.

### Example 4

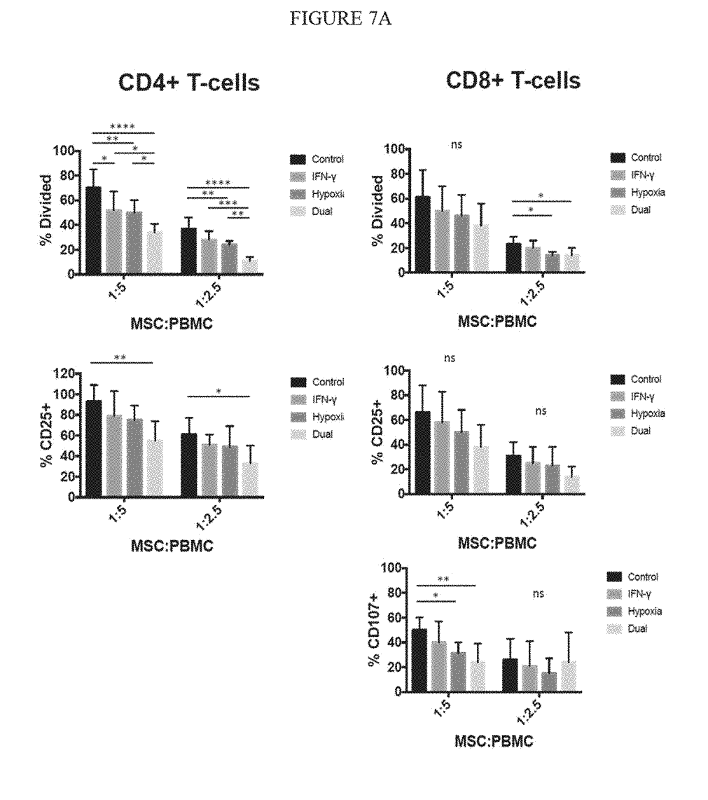

Dual-primed MSCs are superior to single primed MSCs in inhibiting the activation and proliferation of T-cells. FIGS. 7A & 7B indicate how different priming regimens affect the percentages of T cells positive for CD4 and CD8, shown for the indicated ratios between the MSCs and PBMCs on day 5. When MLRs had MSCs in co-culture, MSCs previously kept under control conditions were still able to inhibit T-cell activation (CD25+ expression) and proliferation (% violet negative), but this effect was stronger when they were previously primed with either IFN-γ or hypoxia, and it was the strongest after the cells were exposed to dual priming (FIG. 7A). The inhibitory effect was dose-dependent, with all MSCs providing strongest inhibition when used at the 1:2.5 MSC:PBMC ratio. Group differences were found for both CD4+ and CD8+ T-cells, although they were clearer for CD4+ T-cells. Primed MSCs also better inhibited CD8+ T-cell CD107+ surface expression (measure of cytotoxicity) at the 1:5 ratio, although group differences were no longer present at the 1:2.5 ratio.

Since the baseline inhibitory capacity of the control MSCs depended to some extent on the stimulator-responder PBMC pair used, there were slight shifts in the fraction of divided cells (when normalized to the MLR) for all priming groups across experiments. These shifts led to greater standard deviations for all groups and artificially masked group differences. In order to eliminate the effect of these shifts in baseline MSC inhibition, the data were further normalized to % division of control MSCs for each experiment and then averaged (FIG. 7B). This normalization brought out group differences and made it clear that for creating the most immunosuppressive MSCs, dual priming was superior to single priming, and single priming by either hypoxia or IFN-γ was still superior to no priming. Comparing only single priming regimens, MSC pre-conditioning by hypoxia or IFN-γ were not shown to produce significantly different effects, although the individual experiments suggested a slight advantage of hypoxia.

### Example 5

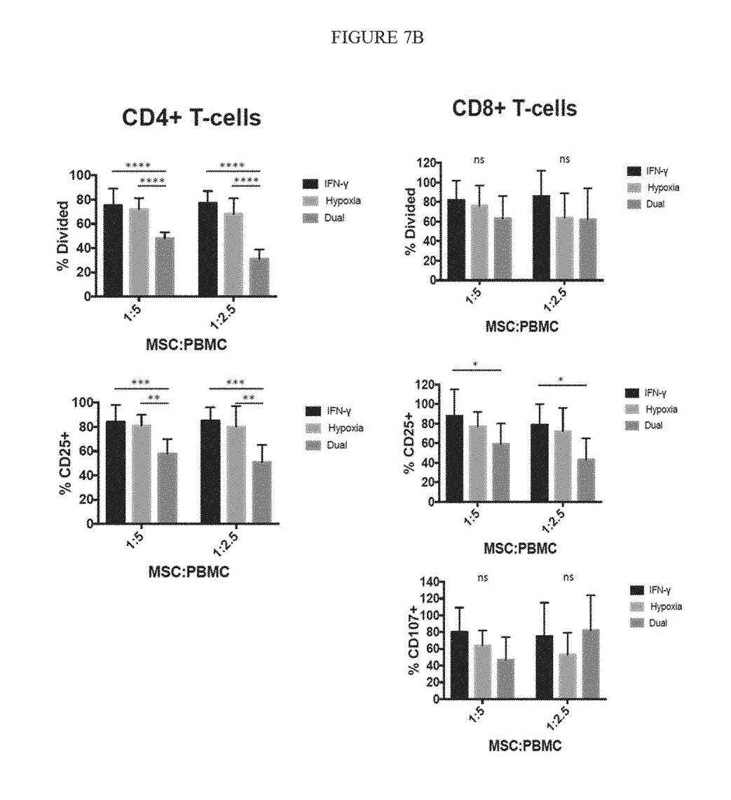

Dual priming shifts T-cells towards a more naive phenotype. To further analyze the effect of differently primed MSCs on T-cell populations, MLRs with either control MSCs or dual-primed MSCs were further evaluated for the expression of CCR7 and CD45RA, to discriminate between naive, memory, and effector T-cell populations (FIG. 8). FIG. 8 shows 1:5 MSC:PBMC ratio on day 5, as evaluated by memory panel markers. The various ratios of naive, central memory, effector memory, and effector T-cells are shown in the quadrants starting at the top right and going counterclockwise. These data are further summarized in stacked bar graphs at the bottom. As expected, a MLR (without MSCs) had fewer naive T-cells than the negative control consisting of responder PBMCs only (without allogeneic stimulus). This loss of the naive fraction corresponded to an increase in the central memory (CM), effector memory (EM), and effector T-cell (ET) populations. MLR co-culture with control MSCs re-shifted the balance to predominantly naive cells. Notably, co-culture with dual-primed MSCs resulted in an even greater fraction of naive T-cells and a shift from effector to central memory cells. Since T-cells are thought to become more activated as they progress from naïve phenotype→CM→EM→ET, dual primed MSCs shifted this balance towards the least activated state.

### Example 6

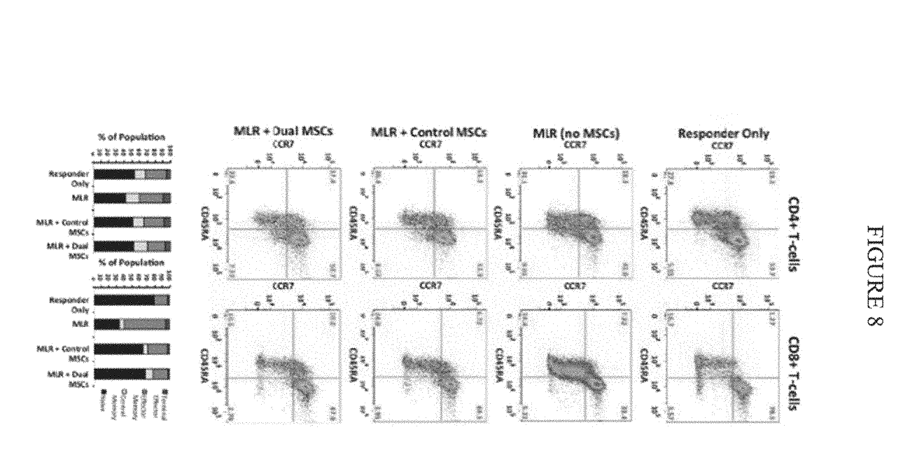

Dual-primed MSCs inhibit the secretion of pro-inflammatory cytokines in mixed lymphocyte reactions. Multiplexed ELISA analysis demonstrated group differences in pro-inflammatory cytokine supernatant levels for MLRs in co-culture with the control, single, or dual-primed MSCs (FIG. 9). In FIG. 9, data are shown for the MSCs that underwent various priming regimens and were co-cultured with MLRs. Quantitative ELISA results are shown for cell supernatant collected an either Day 1 or 3 of the MLR experiment (n=4). While group differences were small or non-significant at Day 1, these differences were more pronounced at Day 3, which reflects the peak cytokine secretion period for several proinflammatory cytokines in MLR experiments. MLRs with control MSCs showed the highest level of pro-inflammatory cytokines (IFN-γ, TNF-α, and IL-1α), sometimes even greater than those measured for the MLR alone. This secretion was greatly dampened by MSC priming, with dual priming leading to the lowest levels of all four cytokines by Day 3.

### Example 7

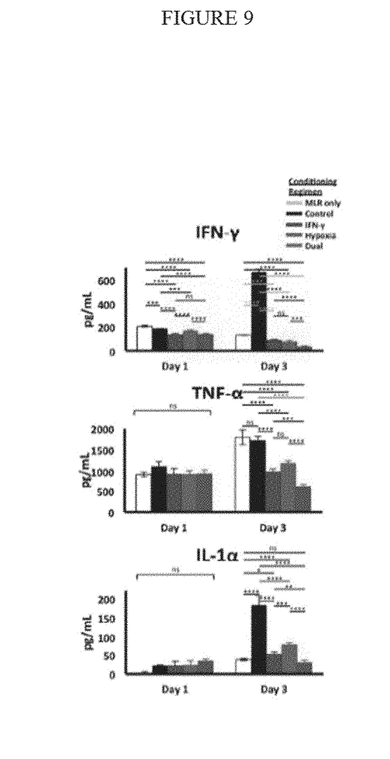

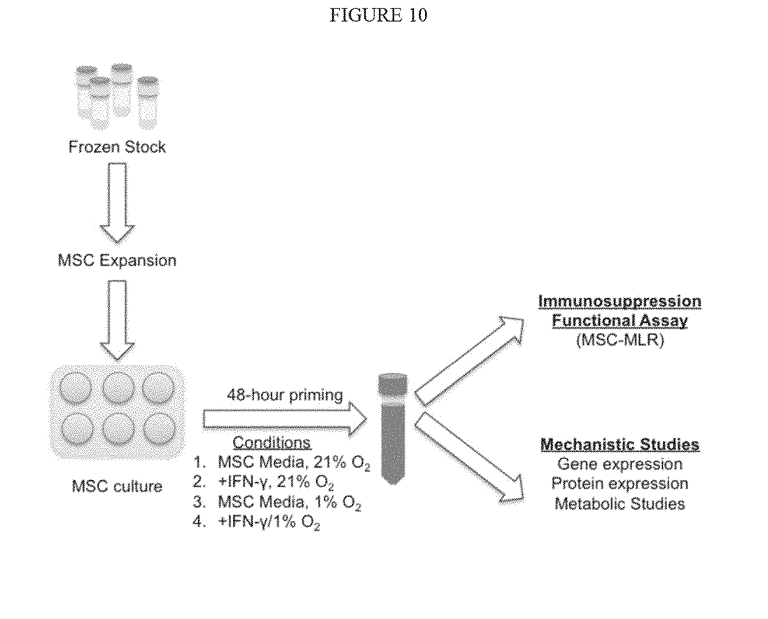

Single priming of MSCs with IFN-γ or hypoxia leads to improvements in T-cell inhibition, while dual priming leads to enhanced immunosuppressive effects. An outline of an experimental approach is shown in FIG. 10. After the optimal durations of priming regimens were determined in pilot studies (48 hours), we tested the efficacy of single and dual priming of MSCs using functional assays for immunosuppression. Addition of control MSCs (cultured in basal medium at normoxia) to mixed lymphocyte reactions (MLRs) resulted in low to moderate inhibition of both CD4+ and CD8+ T cell proliferation (FIG. 11A histograms, violet−) and activation (CD25+; not shown) at Day 6 of MSC-MLR co-cultures. This baseline varied slightly with the donor PBMC pairs used in the MLR, consistent with the known biological variations. Accordingly, the extent of proliferation after co-culture with control MSCs was used as a baseline for comparisons across experiments.

MLRs with MSCs primed with IFN-γ or hypoxia showed similar levels of inhibition and were consistently −25-30% more effective at inhibiting proliferation (violet−) and activation (CD25+) of CD4+ T-cells, and −20% more effective at inhibiting CD8+ T-cells than MLRs with control MSCs (FIG. 11A). In FIG. 11A, the variable inhibition by control and primed MSCs is evident by comparing their % divided population to that of the MLR alone condition (set to 100% division) at day 5. T-cell activation status (% CD25+) and cytotoxic capacity (% CD107+) were similarly determined at day 5. The “responder only” group was a negative control that lacked allogeneic stimulator PBMCs. This inhibitory advantage approximately doubled for MLRs with dual-primed MSCs. Doubling the dose of MSCs by using a 1:2.5 MSC/PBMC ratio led to greater T-cell inhibition regardless of conditioning regimen, whereas the relative inhibitory capacities amongst the priming groups were maintained (FIG. 11A). Inhibition of CD8+ T-cell cytotoxicity (CD107+) has a trend of increase in the presence of MSCs, although the differences between priming groups were not significant (FIG. 11A). In FIG. 11C, all pairwise comparisons were significant except where indicated. Cytokine concentrations for the responder only group were below the detection limit. n=4 p<0.05*, <0.01**, <0.001***, <0.0001****.

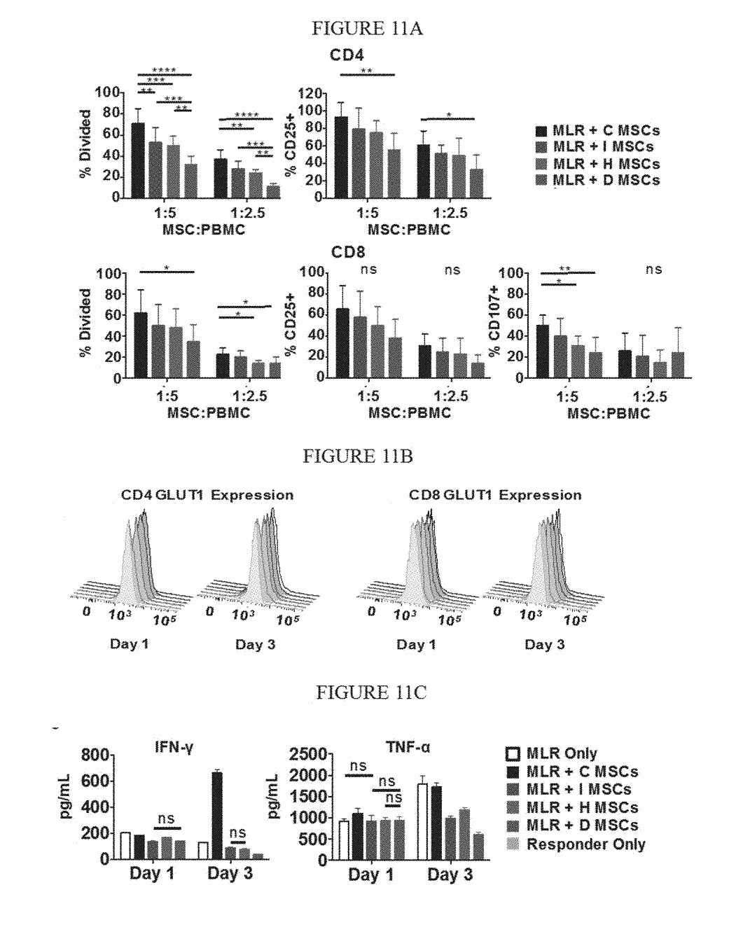

The effect of co-culture on differentiation of the responding T cells in the MLR was studied by analyzing the expression of CCR7 and CD45RA. In FIG. 12, data from a representative experiment are shown for the 1:5 MSC:PBMC ratio on day 5. The various ratios of naïve (N), central memory (CM), effector memory (EM), and terminal effector (TEMRA) T-cells are shown in the quadrants starting at the top right and going counterclockwise. By day 6, the responder-only group (negative control with no allogeneic PBMCs in co-culture with MSCs) had 48% of the CD4+ T cells in the naïve subset (CCR7+CD45RA+), while the MLR (positive control) had only 15.7% cells in this subset. Co-culture with MSCs inhibited the loss of naïve phenotype (FIG. 12), as 24.7%, 31%, 33.5%, and 39.3% of the responding CD4+ T-cells remained in the naïve subset after culture with control MSCs, IFN-γ-primed MSCs, hypoxia-primed MSCs, and dual-primed MSCs, respectively. These differences in the naïve fraction in MSC-MLR co-cultures reflected a shift from the central memory T-cell compartment, as this fraction decreased as the naïve fraction increased.

To investigate how early in the MSC-MLR experiments T-cells exhibited different activation patterns, we looked at two indicators of T-cell activation that preceded the end-point analysis at Day 6 (i.e. % division): GLUT1 expression and pro-inflammatory cytokine levels in the supernatant. The glucose transporter, GLUT1, was upregulated in activated T-cells to fuel glycolysis. As expected, T-cells in the MLR (no MSCs) condition did not upregulate GLUT1 over the responder-only group until Day 3, reflecting slow activation. When MLRs were instead co-cultured with MSCs, there was a rapid increase in T-cell GLUT1 expression even at Day 1. Nevertheless, at Day 1 and Day 3, there were differences depending on the MSC priming, with dual-primed MSCs leading to the lowest T-cell GLUT1 expression (similar to responder-only T-cell levels), although single-primed MSCs still led to less T-cell GLUT1 expression than the control MSCs (FIG. 11B). Differences in pro-inflammatory cytokine levels were only apparent by Day 3, and MSC-MLRs with dual-primed MSCs had lower levels than single-primed MSCs (FIG. 11C). Curiously, control MSCs led to higher supernatant concentrations of pro-inflammatory cytokines than even the MLR (no MSCs) condition, suggestive of an initial allogeneicity. However, the control MSCs were still inhibitory in MLRs, likely due to an immunosuppressive reactivity to local pro-inflammatory cytokines.

### Example 8

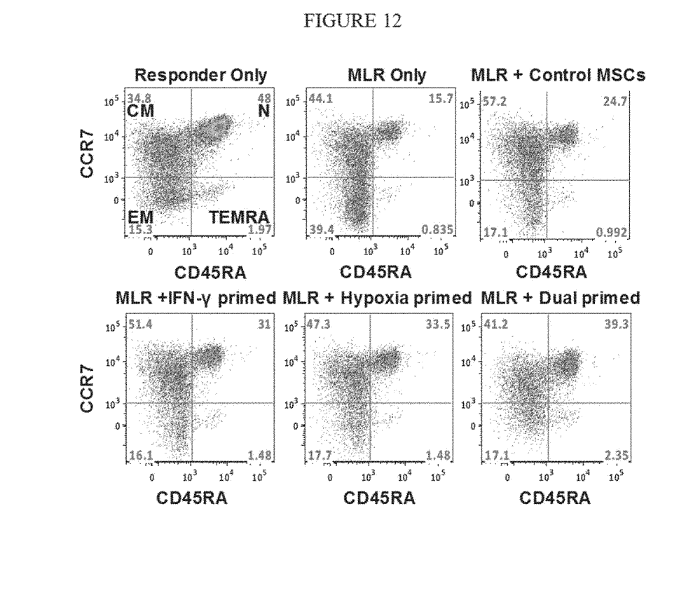

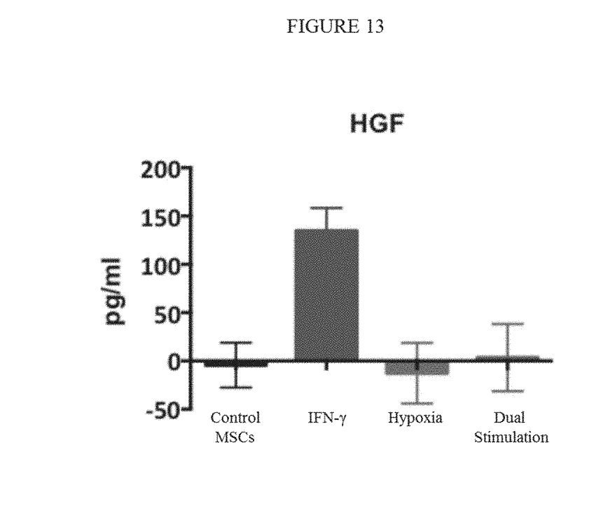

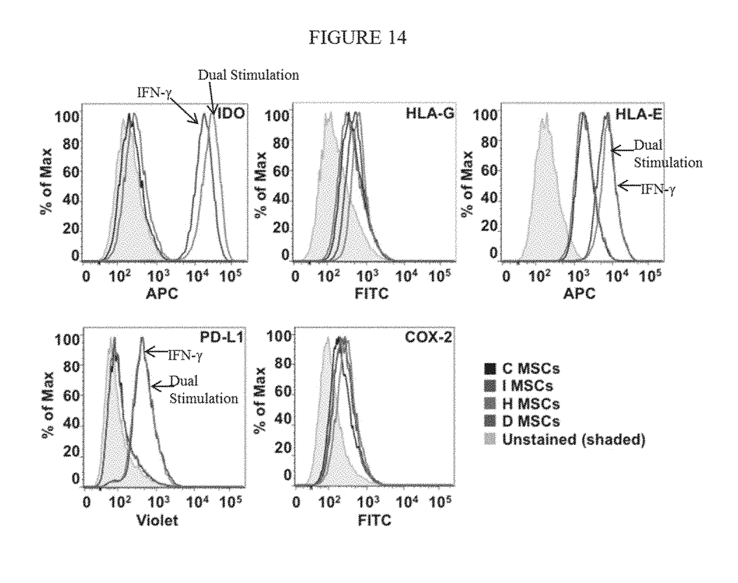

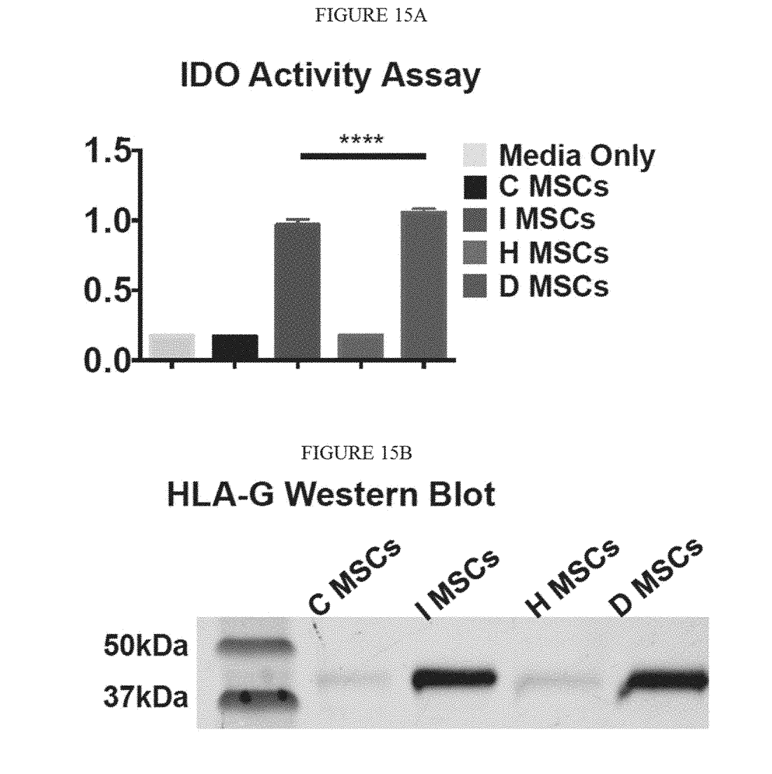

Genes upregulated by dual priming at mRNA level show strong induction at protein level. We next investigated the mRNA trends from MSC priming at the protein level. Some trends were confirmed. For example, HGF protein was only detectable in the supernatant of IFN-γ-primed cells, (FIG. 13). Notably, PD-L1, IDO, and HLA-E, which had less mRNA induction from dual priming than from IFN-priming, showed similar or greater induction by dual priming (FIG. 16 and TABLE 3). In FIG. 14, histograms are shown from a representative experiment with 20 000 events (same experiment conducted n=3 times). All pairwise comparisons for IDO, HLA-G, HLA-E, and PD-L1 were significant except control vs. hypoxia (p<0.0001). By contrast, only control vs. hypoxia was significant for COX-2 (p<0.01). In fact, IDO protein expression was significantly greater after dual priming than after exposure to IFN-γ alone. This unanticipated result was confirmed by an IDO activity assay (FIG. 15A), where dual-primed MSCs showed significant enhancement of IDO activity over IFN-γ-primed MSCs, consistent with the flow cytometry data. In FIG. 15A, primed cells were replated into 96-well plates at 10 000 cells per well for assessment of overnight IDO activity, which corresponds to detecting the tryptophan byproduct kynurenine via absorbance at 480 nm. 6 wells were averaged per condition.

Enhancement of the two genes higher induced by hypoxia at the mRNA level (COX-2 and HLA-G) was also confirmed by flow cytometry. However, COX-2 protein expression was equally enhanced by all priming regimens, and protein levels were only slightly greater than those for control MSCs. While MSC HLA-G protein levels were the greatest after dual priming, consistent with the PCR data, protein levels were greater from priming by IFN-γ than by hypoxia, opposite to the PCR findings. This result was confirmed by Western blot analysis, which shows that HLA-G was substantively induced by both IFN-γ and dual priming at the protein level (FIG. 15B). FIG. 15B shows a Western blot where each lane was initially loaded with the same amount of protein (BCA assay). P<0.0001****. Overall, hypoxia did not upregulate any of the studied proteins to a greater extent than IFN-γ.

### Example 9

Hypoxia priming and dual IFN-γ/hypoxia priming shift metabolism from oxidative phosphorylation towards glycolysis. Since it was unclear from protein level studies why hypoxia priming of MSCs led to a similar level of MLR inhibition as IFN-γ priming, metabolic studies were pursued. Seahorse assay results Show that hypoxic priming of MSCs shifts them away from oxidative metabolism (lower oxygen consumption rate) towards glycolysis (higher extracellular acidification rate, ECAR) (FIG. 16A). This finding was supported by the analysis of glucose levels of Day 1 and Day 3 MLR-MSC co-culture supernatant, which showed the lowest glucose levels for MLRs with hypoxia and dual-primed MSCs (FIG. 16B; TABLE 4).

TABLE 4
Daily glucose consumption and lactate production in MSC-
MLR experiments.

| | Daily Glucose | | Daily | |
|---|---|---|---|---|
| | Consumption | | Lactate Production | |
| | Day 1 | Day 2-3 Avg | Day 1 | Day 2-3 Avg |
| Responder Only | 3.65 | 8.53 | <1 | 1.97 |
| MLR Only | 6.80 | 15.38 | <1 | 2.92 |
| MLR + C MSCs | 34.45 | 40.33 | 5.61 | 7.85 |
| MLR + I MSCs | 46.10 | 40.45 | 6.88 | 7.36 |
| MLR + H MSCs | 61.55 | 39.50 | 9.22 | 5.53 |
| MLR + D MSCs | 70.85 | 45.60 | 14.08 | 6.30 |

We explored if the immunosuppressive phenotype of MSCs can be promoted by cell priming using two microenvironmental cues: IFN-γ and hypoxia, which are present in a number of conditions associated with immune tolerance. Our goal was three-fold: (i) to compare the effects of IFN-γ and hypoxia on MSCs under otherwise similar conditions, (ii) to determine if the concurrent application of the two priming cues can promote immunosuppressive phenotype in MSCs beyond levels achievable with either factor alone, and (iii) identify immunosuppressive mechanisms promoted by MSC priming.

Based on the PCR data, a 48-hour priming regimen was chosen, and the two genes that became most highly upregulated by IFN-γ (IDO and PD-L1) and hypoxia (HLA-G and COX-2) were further studied. The analysis of transcription kinetics demonstrated that, with the exception of COX-2, these genes stay upregulated for one week after removal from priming conditions and could be “boosted” once re-exposed to the same priming regimens. This finding is important as it suggests that even though MSCs are taken out of priming conditions for administration to a patient, changes in expression will not be lost, and the cells maintain their ability to respond to inflammatory and hypoxic cues in vivo.

To demonstrate the functional significance of these changes in expression, we evaluated the control (not primed), single-primed, and dual-primed MSCs in MLR co-culture experiments, after the cells were removed from their priming conditions and co-cultured with allogeneic PBMCs. It is not surprising that the MSCs cultured under control conditions were able to attenuate the MLR response, since activation of T-cells eventually leads to the release of pro-inflammatory cytokines, which can induce immunosuppressive behavior in “control MSCs”.

Single and dual priming regimens clearly augmented the immunosuppressive capacity of MSCs in MLR co-culture experiments, and these effects were shown using a range of lymphocyte donors. There were no significant differences in MSC-based T-cell suppression for single priming by IFN-γ and hypoxia. However, these two regimens have different practical value for clinical application. Since cell therapies use hundreds of millions of cells, the cost of an IFN-γ based regimen increases in proportion with cell number, whereas a hypoxia-based strategy would only require the use of a hypoxic incubator. Combining the two stimuli clearly resulted in stronger immunosuppressive effects than the use of either stimulus alone. We document this finding using multiple assays including the inhibition of T-cell proliferation, T-cell activation, CD8+ T-cell cytotoxicity, and pro-inflammatory cytokine secretion. Dual-primed MSCs further shifted T-cell populations towards more naive and quiescent cell type.

From the PCR data, one might explain the observed superiority of dual priming based on IFN-γ upregulating IDO and PD-L1, and hypoxia upregulating COX-2 and HLA-G. This explanation is challenged by the flow cytometry data for immunosuppressive proteins. While it is true that dual priming led to expression of all of these factors at the protein level, IFN-γ alone led to almost equivalent protein levels. The small increase in IDO from dual priming over IFN-γ single priming is suggestive of an interaction between the hypoxia and IFN-γ induced pathways, making cells more susceptible to IFN-γ signaling.

The flow cytometry data do not explain why hypoxia single priming was so effective, leading to MSCs that were as immunosuppressive as those primed with IFN-γ. As per flow cytometry for surface markers, hypoxia was only mildly superior to IFN-γ in terms of COX-2 expression, but it led to less induction than IFN-γ for all three other proteins. Admittedly, only a subset of genes and proteins were analyzed extensively in our studies, and hypoxia could be upregulating some other immunosuppressive protein. It should be noted, however, that the other genes implicated in immunosuppression in the initial PCR screen, either did not change from either stimulus, or they were induced by IFN-γ but not hypoxia (i.e. iNOS, HGF, and HLA-E).

Since no compelling reason for the functional effect of hypoxic-priming was offered by protein expression studies, we started to explore metabolic changes. Naive T-cells have low metabolic requirements, which they fulfill via oxidative phosphorylation (OXPHOS). However, upon activation, they switch to being highly metabolic via aerobic glycolysis. While this generates fewer ATP per glucose than OXPHOS, it provides the ATP more quickly and leads to the production of important biomolecules for the anabolic metabolism needed to sustain cell proliferation. Due to this switch to aerobic glycolysis, proliferating T-cells become highly glucose dependent.

Tumors can inhibit immune cell attack by out-competing T-cells for nutrients. Tumors also use glycolysis, and the more glycolytic the tumor, the more it can inhibit effector T-cells via depleting glucose and producing lactate. These changes in environmental nutrients influence signaling through the mechanistic-target-of-rapamycin (mTOR) pathway, such that T-cells do not differentiate into activated (and dividing) effector cells. We further hypothesized that MSCs exposed to hypoxia (either alone or in the dual-priming regimen) would switch from oxygen-dependent OXPHOS to glycolysis. We demonstrated this by Seahorse assay and glucose measurements for MLRs on Days 1 and 3, which is the period of time when T-cells are becoming activated but not yet dividing (as confirmed by the presence of only non-divided populations on flow cytometry).

Hypoxia primed MSCs consume much more glucose and can thereby influence cell fate decisions of initially naive lymphocytes over the first three days of the MLR. Since 50 kiL of fresh media were provided to the cell cultures (Day 3 samples for glucose analysis were taken prior to medium addition), any activated T-cells would have had fresh nutrients to fuel division. However, if fewer surrounding nutrients had already influenced their activation and differentiation to effector T-cells, they still would not have proliferated. This could also explain why hypoxia was even more effective at inhibiting CD8+ T-cell proliferation. While CD4+ T-cells have been shown to increase OXPHOS along with glycolysis upon activation, this has not been shown for CD8+ T-cells, and the higher CD8+ T-cell glycolytic flux may make them more susceptible to inhibition by glucose deprivation. The switch towards glycolysis from hypoxic exposure of MSCs may have other implications as well. Along with upregulation of angiogenic factors, this switch may help explain why hypoxia primed MSCs survive better in ischemic environments in several animal models. It may also imply that it would be beneficial to use the cells themselves over their secreted products in any kind of therapy.

An important conclusion from this study is that a single dose of MSCs would be more impactful in treating an acute condition than a chronic condition, in which there is ongoing immune cell-activation.

### Example 10

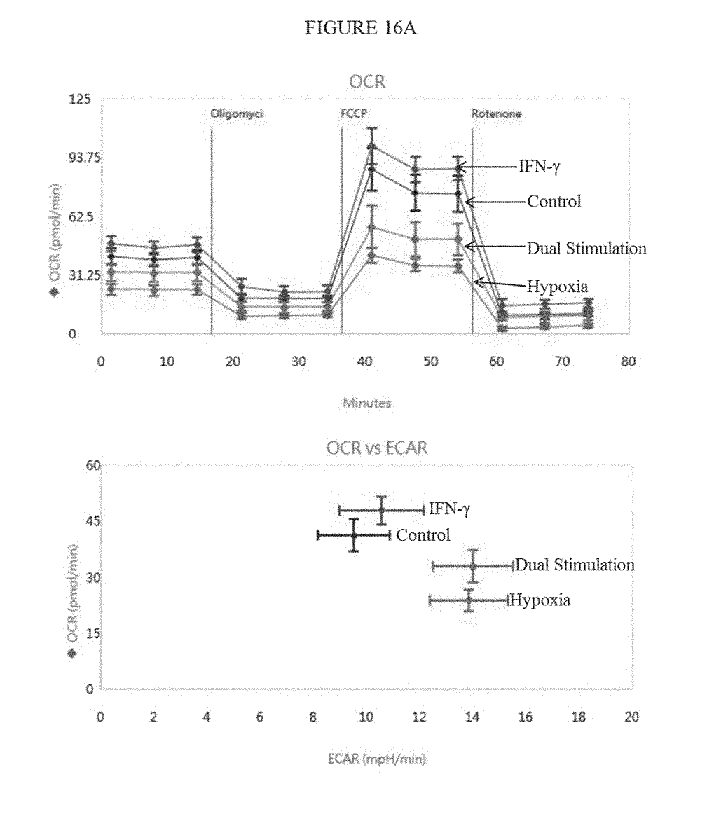

Hypoxia and dual priming induce metabolic shift to glycolysis with rapid lactate production. We next explored possible metabolic explanations for hypoxia-based immunosuppression by investigating how different priming regimens affected major metabolic pathways in MSCs. IFN-γ-primed MSCs had the highest oxygen consumption rate (OCR, by Seahorse Analysis), which is considered to be a marker for oxidative phosphorylation (OXPHOS). The OCR decreased from control MSCs to dual-primed MSCs and was lowest for hypoxia-primed MSCs (FIG. 17A). Importantly, the reduced OCR in hypoxia-primed and dual-primed MSCs correlated with enhanced glycolytic metabolism, as indicated by increases in extracellular acidification rate (ECAR) for these two groups (FIG. 17A). The shift towards glycolysis in hypoxia-primed and dual-primed MSCs was further supported by unique upregulation of GLUT1, the inducible glucose transporter also required for T-cell glycolysis (FIG. 17B).

We then investigated if the observed metabolic changes could explain the trends seen in the MSC-MLR co-cultures (FIGS. 11A-11C), specifically (i) why hypoxia-primed MSCs were as inhibitory as IFN-γ-primed MSCs and (ii) if metabolic changes could explain the enhanced immunosuppressive efficacy of dual-primed MSCs. Glucose and lactate levels were measured at Days 1 and 3, time points that precede T-cell division (to maintain consistent cell numbers amongst groups) but during which T cells may become activated. As expected, by Day 3, activated PBMCs in the MLR (no MSCs) condition showed higher glucose consumption and lactate production than the responder only group (FIG. 17C). In FIGS. 17A-17C, all pairwise comparisons are significant at p<0.001 except where indicated.

Overall, MSCs had large influence on the metabolic environment. Dual-primed and hypoxia-primed MSCs led to the greatest glucose depletion and lactate production by Day 1, consistent with higher GLUT1 expression and induction of glycolysis. Notably, dual-primed and hypoxia-primed MSCs led to a 3-fold and 2-fold (˜15 mM and 10 mM vs. 5 mM), respectively, increase of lactate in the supernatant relative to control MSCs. Glucose levels continued to decline between Days 1 and 3, while the rate of consumption during this phase was similar amongst MSC-MLR groups (drop of ˜40 mg/dl for control, IFN-γ and hypoxia-primed MSC-MLR co-cultures; 45 mg/dl for dual-primed; TABLE 5). Lactate accumulation started to plateau between Days 1 and 3, although the highest levels were still found in MSC-MLR reactions with dual-primed MSCs.

TABLE 5
Mean fluorescence intensity ± SD for data shown in FIG. 14.

| | IDO | HLA-G | HLA-E | COX-2 | PD-L1 |
|---|---|---|---|---|---|
| C MSCs | 299 ± 227 | 564 ± 570 | 2396 ± 3640 | 310 ± 2003 | 169 ± 288 |
| I MSCs | 20 900 ± 11 087 | 668 ± 680 | 8848 ± 7296 | 337 ± 796 | 611 ± 489 |
| H MSCs | 482 ± 2405 | 554 ± 797 | 2375 ± 4580 | 358 ± 1222 | 167 ± 265 |
| D MSCS | 31 766 ± 16 677 | 796 ± 1212 | 9721 ± 6944 | 336 ± 827 | 575 ± 435 |

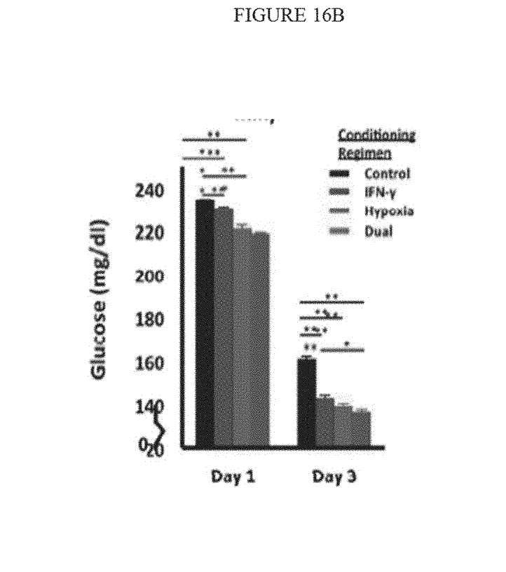

To explore the consequences of high extracellular lactate concentrations, we looked at their effects on intracellular pH and the proliferation of T-cells. Increasing extracellular lactic acid levels in fresh complete AIM-V media (please see Methods) led to a dose-dependent drop in T-cell pHi, which started to become pronounced at 15 mM, and by 30 mM dropped dramatically along with media pH (FIG. 18A). As the lactic acid concentration increased from 20 mM to 30 mM, there was also a notable change in the scatter properties of the T-cells, consistent with cells undergoing apoptosis (FIG. 18B). Titrating lactic acid into media also attenuated both CD4+ and CD8+ T-cell division in response to the strong mitogen Concanavalin A (ConA) (FIG. 18C). T-cell division was reduced to 30-45% of the maximum rate, and at the lactate concentration of 15 mM, T-cell division was almost completely eliminated (FIG. 18C).

In summary, the results demonstrate that IFN-γ and hypoxia, elicit distinctly different mechanisms of immune suppression in MSCs. We show that combining these separate pathways by exposure of MSCs to both priming cues leads to enhanced immunosuppressive effects. While IFN-γ is the most frequently studied MSC priming regimen, we believe that hypoxia is a relatively low cost addition that not only promotes glycolysis, but also enhances the IFN-γ induced expression of IDO, HLA-G, and HLA-E. Priming MSCs in this manner should lead to a more significant “hit” in the hit-and-run paradigm of MSC-immunomodulation and could promote a more efficacious therapy for treatment of dozens of autoimmune and inflammatory disorders.

### Example 11

Mass spectrometry confirms that hypoxia influences MSC metabolism but does not upregulate proteins with direct immunosuppressive capacity. To further evaluate our metabolic hypothesis for hypoxia-induced MSC immunosuppression and for why dual-primed MSCs showed enhanced improvements in immunosuppression compared to single priming, mass spectrometry was performed. This was to confirm that cells exposed to hypoxia did not upregulate any proteins with immunosuppressive capacity that were missed on PCR or flow cytometry analysis.

Proteins that changed in expression due to hypoxia priming were predominantly mitochondrial proteins that had a role in cellular metabolism. Specifically, proteins involved in oxidative phosphorylation and the TCA cycle were down-regulated, as were mitochondrial ribosomal proteins. When STRING analysis was performed on the list of proteins that changed over 2-fold from hypoxia priming (over control MSCs), it did not associate any proteins with immunomodulation.

Dual primed cells showed similar changes in the metabolic pathways altered by hypoxia alone, although in some instances, a protein was downregulated slightly less than 2-fold such that there was not 100% overlap in the list of downregulated proteins between hypoxia and dual primed cells (TABLE 6).

TABLE 6
STRING analysis of pathways altered by hypoxia primed or
dual primed MSCs compare to control MSCs.

| | | | |
|---|---|---|---|
| | | Protein | False discovery |
| #pathway ID | Pathway description | count | rate |

Hypoxia primed

| | | | |
|---|---|---|---|
| GO.0032543 | Mitochondrial translation | 18 | 4.93E−16 |
| GO.0006119 | Oxidative phosphorylation | 16 | 1.52E−16 |
| GO.00072350 | Tricarboxylic acid metabolic | 7 | 2.27E−06 |
| | process | | |

Dual Primed

| | | | |
|---|---|---|---|
| GO.0032543 | Mitochondrial translation | 16 | 2.79E−07 |
| GO.0006119 | Oxidative phosphorylation | 11 | 3.08E−05 |
| GO.00072350 | Tricarboxylic acid metabolic | 7 | 6.20E−04 |
| | process | | |

### Example 12

Mesenchymal stromal cells (MSCs) are being studied as a therapy for autoimmune and inflammatory disorders due to their capacity for immunosuppression. Depending on the pathology, they are introduced intravascularly or locally. In both cases, they become immunosuppressive only in response to specific environmental cues. We recently compared the effect of IFN-γ priming to hypoxia priming on the immunosuppressive capacity of MSCs in vitro. We found that MSCs primed by IFN-γ (100 ng/mL) or hypoxia (1% O₂) alone were equally efficacious at inhibiting CD4 and CD8 T-cell division in mixed lymphocyte reactions (MLRs), whereas combining these two cues led to MSCs that were twice as immunosuppressive. While IFN-γ clearly upregulated immunosuppressive proteins (IDO, PD-L1, HLA-G, HLA-E), hypoxia did not. We thus investigated possible immunometabolic mechanisms of hypoxia-based inhibition. MSC exposure to hypoxia resulted in a shift towards glycolytic metabolism (Seahorse Assay), an increase in GLUT1 expression (flow cytometry), and faster glucose consumption and lactic acid production in MSC-MLR co-cultures. In fact, by Day 1 of MSC-MLR co-culture, extracellular lactate had already reached levels that could inhibit T-cell division when hypoxia-exposed MSCs were incorporated. We thus propose that hypoxia priming may provide added benefits to IFN-γ priming for locally implanted MSCs, where the MSCs may be able to dominate the nearby metabolic environment to provide another mechanism for immune suppression.

### Example 13

We used a polymer method to isolate exosomes from the supernatant of unprimed MSCs or those primed with 48-hours of combined hypoxia/IFN-γ. Other potential methods of isolating exosomes from the supernatant of MSCs include immunoprecipitation, ultracentrifugation, and size exclusion chromatography. There were consistently 2-4× more exosomes secreted from primed MSCs. We labeled these exosomes and dosed them into mixed lymphocyte reactions (MLRs) at concentrations of 10, 50, 125 and 250 μg/mL. This showed dose-dependent uptake of the exosomes into mononuclear cells (PBMCs), but the uptake was less profound when exosomes came from primed MSCs. While no division occurred when the exosomes were added to only responder PBMCs, in a full MLR, the exosomes attenuated T-cell division in a dose-dependent manner. Doses >50 μg/mL exacerbated T-cell division from both exosome sources, but exosomes from primed MSCs were able to inhibit T-cell division once the concentration was lowered to 50 μg/mL. We are still investigating the mechanisms of this dose and source-dependent attenuation, but it is clear that monocytes (not T-cells) predominantly take up the exosomes.

To test this hypothesis about the mechanism of action involved in MSC priming, passage 5 MSCs were grown to confluency in 6-well plates and exposed to stimulation by IFN-γ (100 ng/mL Peprotech) or hypoxia (37° C., 5% CO₂, 1% O₂), dual stimulation (IFN-γ and hypoxia), or control conditions (normoxia; basic MSC media) for 48 hours. Subsequently, a polymer-based ExoQuick TC reagent was used to isolate exosomes from 5 mL of cell culture supernatant from each condition. Isolated particles were positive for the CD63 tetraspanin exosome surface marker, as determined by capturing fluorescently labeled particles on anti-CD63 magnetic microbeads and flow cytometry. Other potential markers to identify exosomes are CD9 and CD81. The concentration and size distribution of exosomes isolated at various MSC culture conditions were measured by NanoSight analysis. The uptake of exosomes by peripheral blood mononuclear cells (PBMCs) was verified by first labeling the exosomes with a DiI reagent (25 μg/ml), removing the excess reagent via spin columns, and adding the exosomes to activated PBMC culture. After 24 hours, internalization of exosomes by PBMCs was examined by flow cytometry analysis.

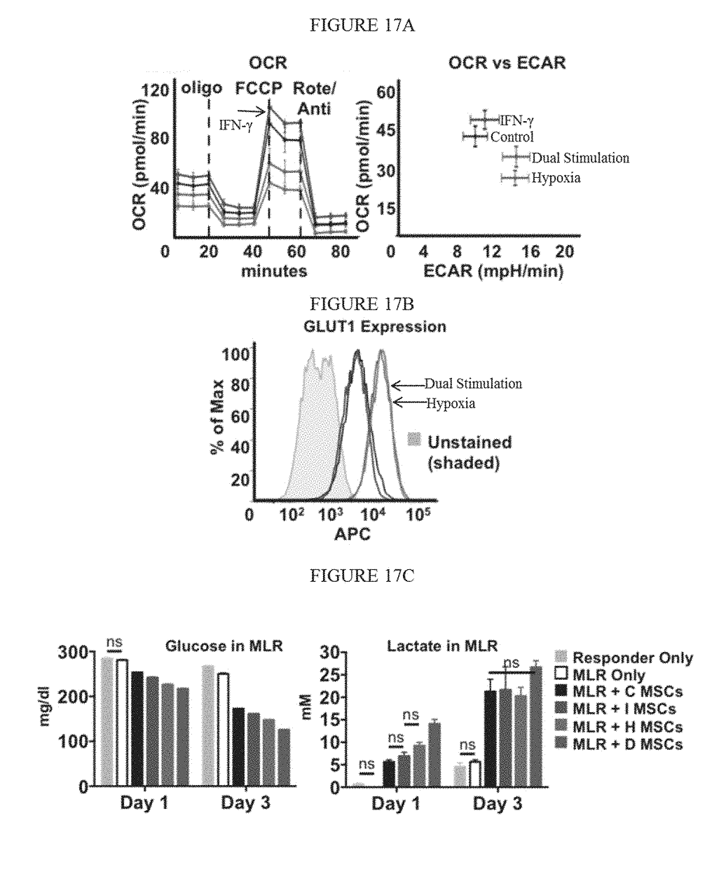

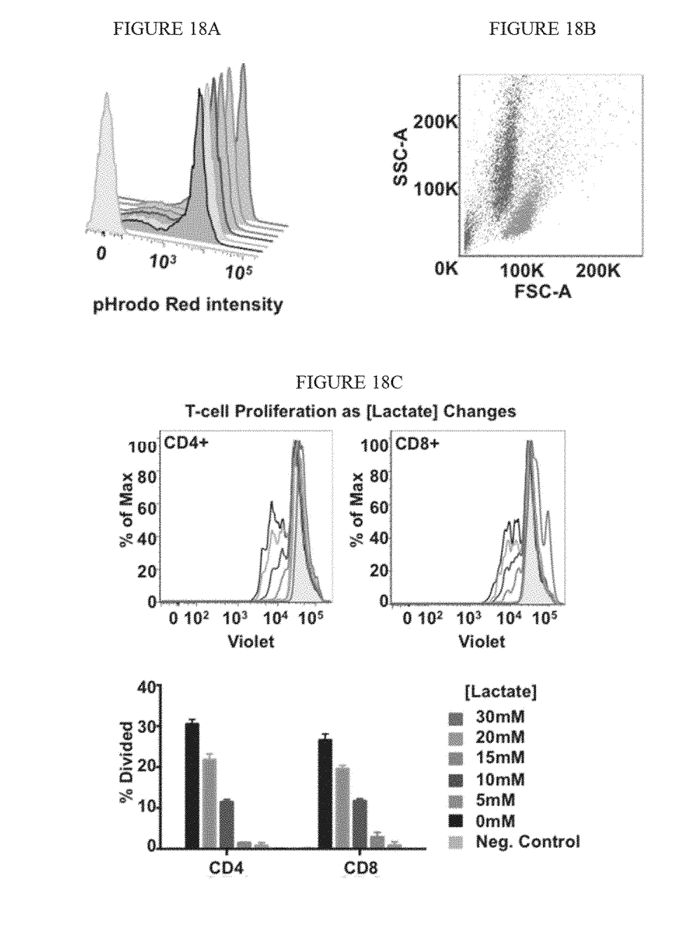

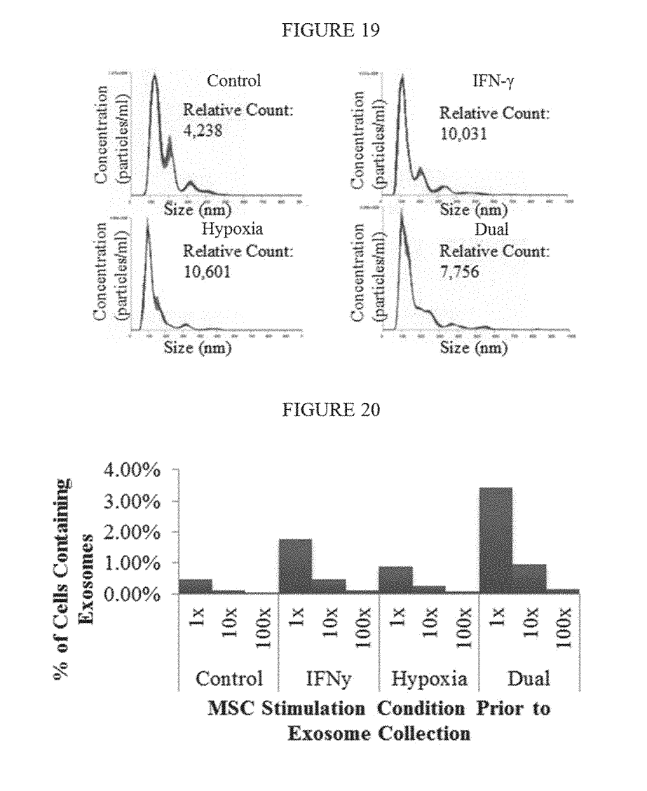

The amounts of secreted exosomes (normalized to the number of MSCs) were 1.8-2.5× higher when MSCs were subjected to IFN-γ and/or hypoxia stimulation, as compared to control culture (FIG. 19). MSC culture conditions also affected the size distribution of exosomes, with most prominent differences around the 200 nm and 550 nm diameters (FIG. 19). Dose-dependent exosome internalization was observed within activated PBMCs 24 hours after exosome addition for all MSC-derived exosome populations (FIG. 20).

We report that CD63+ exosomes from differentially stimulated MSCs differ in population concentration and size distribution, and are internalized in PBMCs in a dose-dependent manner. These differences may inform our understanding of immunomodulation by stimulated MSCs, as different stimuli may activate MSCs to secrete specific immunomodulatory exosomes. To understand the nature of these differences, next-generation sequencing of the exosome RNA content is being conducted, and sequence alignments will enable identification of MSC culture condition-specific RNA cargo. For functional relevance, the effect of exosomes on T-cell proliferation is also being studied in mixed lymphocyte reactions, with primed exosomes in concentrations of 1 μg/mL to 50 μg/mL.

### Example 14

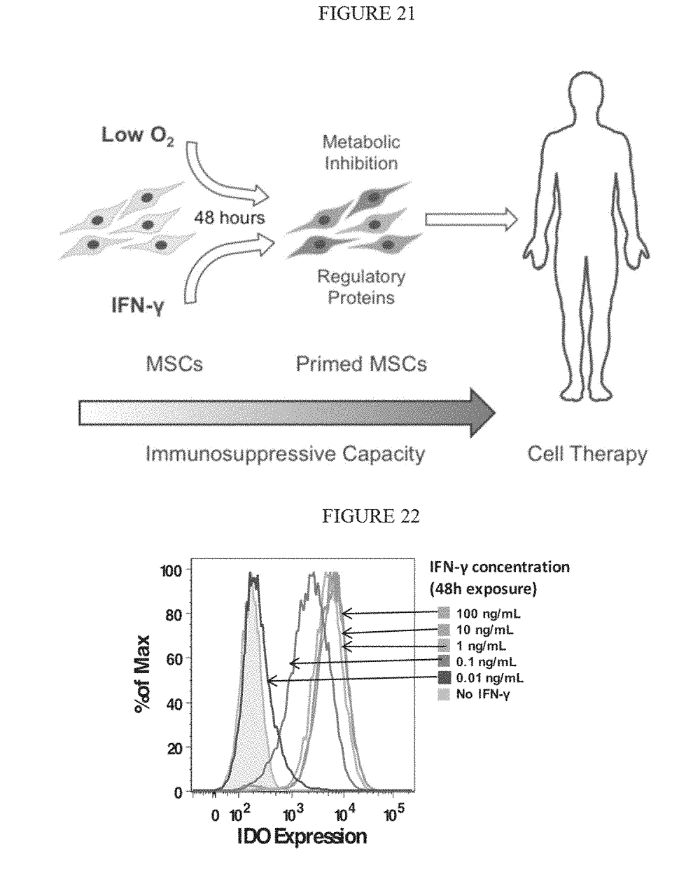

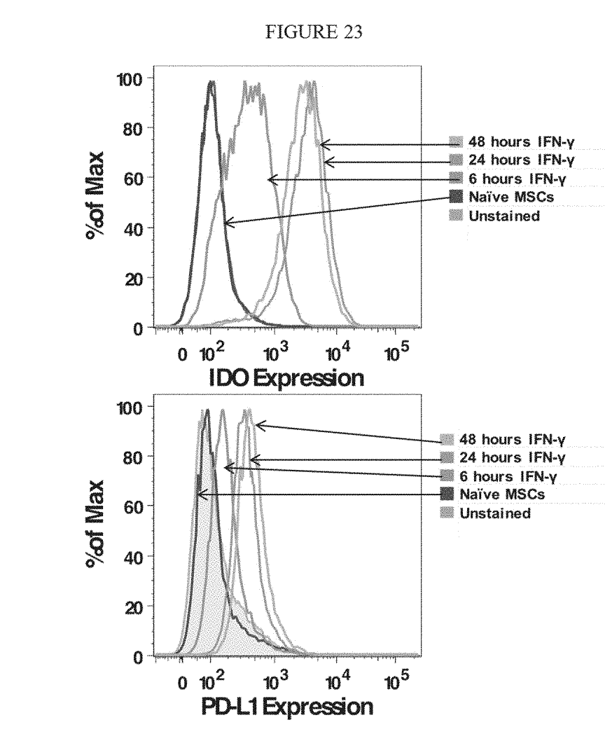

Titration studies were carried out to determine whether IFN-γ at different concentrations or for different time periods had differential effects on MSC expression of IDO and/or PD-L1 (FIGS. 22 & 23). FIG. 22 shows that IDO requires at least 1 ng/mL of IFN-γ for maximum induction after 48 hours of exposure, although even 0.1 ng/mL can still lead to significant induction. Thus, the IFN-γ dose could be used in a range of 0.1 ng/mL to 100 ng/mL for future animal and human studies, while still reproducing the same beneficial phenotype, with the lower end of that range more likely to mimic physiologic conditions in humans. FIG. 23 shows that while 48 hours are required for maximum IDO and PD-L1 expression, IDO and PD-L1 are already upregulated after only 6 hours of exposure. Therefore, priming of MSCs may be carried out for less than 48 hours to achieve biological effects.

### Example 15

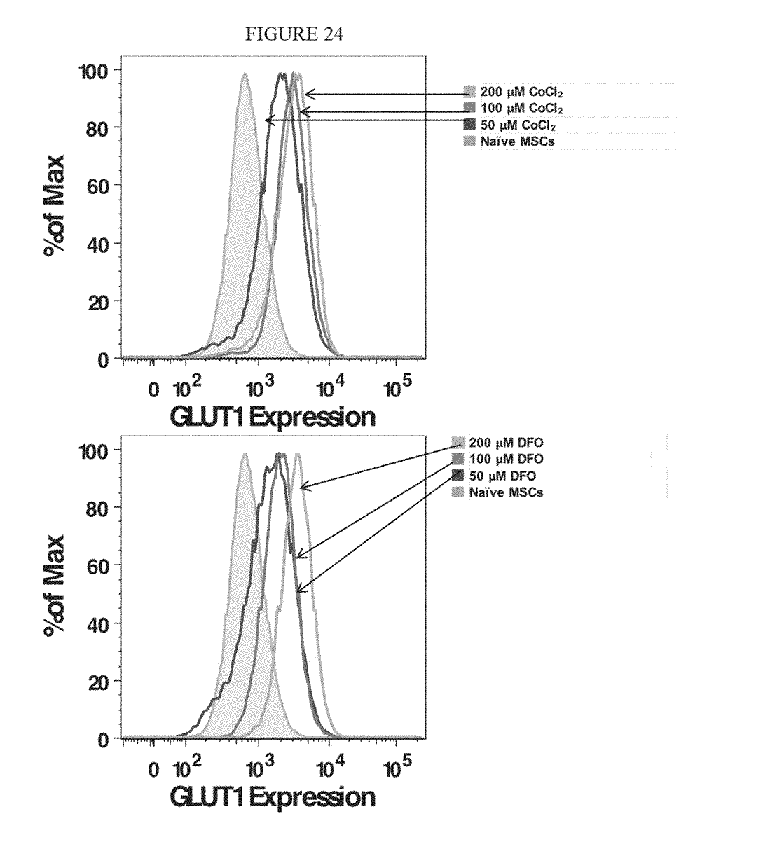

Studies were carried out to determine the effect of hypoxia mimicking agents CoCl₂ and DFO on MSCs (FIG. 24). FIG. 24 shows that CoCl₂ and DFO are both able to upregulate GLUT 1, which is a metric for the hypoxia pathway, where it indicates a metabolic switch to glycolysis. Since we believe this metabolic switch is one of the main ways by which hypoxia preconditioning enables MSCs to inhibit immune cells, the data shown in FIG. 24 suggests that hypoxia-mimicking agents at concentrations of 50 μM to 200 μM could be substituted for hypoxic conditions of 37° C., 5% CO₂, and 1%-5% O₂ in our priming regimen.

What is claimed is:

1. A method for preparing immunosuppressive primed mesenchymal stromal cells comprising:
obtaining unprimed mesenchymal stromal cells isolated from a source; and
applying a pro-inflammatory cytokine to the mesenchymal stromal cells in a hypoxic culture condition in vitro.

2. The method of claim 1, wherein the source is selected from the group consisting of adipose tissue, umbilical cord, bone marrow, gingiva, and iPSCs.

3. The method of claim 1, wherein the mesenchymal stromal cells are exposed to the hypoxic culture condition for 1 hour to 48 hours.

4. The method of claim 1, wherein the pro-inflammatory cytokine is selected from the group consisting of IL-1α, IL-IB, TNF-α, IFN-γ, IL-6, IL-12, IL-17, and IL-23.

5. The method of claim 4, wherein the pro-inflammatory cytokine is IFN-γ.

6. The method of claim 5, wherein IFN-γ is at a concentration of 0.1 ng/mL to 100 ng/mL.

7. The method of claim 1, wherein the hypoxic culture condition comprises exposing the mesenchymal stromal cells to 37° C., 5% CO₂, and 1% O₂ to 5% O₂.

8. The method of claim 1, wherein the hypoxic culture condition comprises exposing the mesenchymal stromal cells to a hypoxia mimetic.

9. The method of claim 8, wherein the hypoxia mimetic is selected from the group consisting of desferoxamine, cobalt chloride, hydralazine, nickel chloride, diazoxide, and dimethyloxalyglycine.

10. The method of claim 9, wherein the hypoxia mimetic is at a concentration of 50 μM to 200 μM.

11. The method of claim 1, wherein the hypoxic culture condition is created by application of hypoxia-inducing factor.

12. The method of claim 1, further comprising the step of isolating exosomes secreted from the mesenchymal stromal cells following exposure to the proinflammatory cytokine and the hypoxic culture condition.

13. A method for treating a subject experiencing a condition or preventing a condition in a subject at risk for the condition, wherein the condition is selected from the group consisting of cytokine storm, sepsis, autoimmune disease, transplant rejection, graft-vs-host disease, acute tissue injury, diabetic ulcer, and inflammatory disease; and wherein the method comprises administering a primed mesenchymal stromal cell prepared according to claim 1 to the subject experiencing the condition or at risk for the condition.

14. The method of claim 13, further comprising administering an immunosuppressive agent to the subject.

15. The method of claim 14, wherein the immunosuppressive agent is selected from the group consisting of calcineurin inhibitors, steroids, microphenolate mofetil, anti-CD3 antibodies, aziothioprine, cyclophosphamide, ifosfamide, and monoclonal antibodies used for immunosuppression.

16. The method of claim 13, further comprising administering an immunotherapy to the subject.

17. The method of claim 16, wherein the immunotherapy comprises chimeric antigen receptor T-cells.

18. A composition comprising primed mesenchymal stromal cells prepared by applying a pro-inflammatory cytokine to mesenchymal stromal cells in a hypoxic culture condition according to claim 1.

19. The composition of claim 18 wherein the primed mesenchymal stromal cells are in a pharmaceutically acceptable carrier.


SEQUENCE LISTING (32 sequences)

SEQ ID NO: 1 - DNA, 21 bp, Artificial Sequence
  Description of Artificial Sequence Synthetic primer
aaggtgaaggtcggagtcaac

SEQ ID NO: 2 - DNA, 19 bp, Artificial Sequence
  Description of Artificial Sequence Synthetic primer
gaagaggagacacggaaca

SEQ ID NO: 3 - DNA, 20 bp, Artificial Sequence
  Description of Artificial Sequence Synthetic primer
atggaaccctccttttactc

SEQ ID NO: 4 - DNA, 19 bp, Artificial Sequence
  Description of Artificial Sequence Synthetic primer
ggtgaccaaactcctgcca

SEQ ID NO: 5 - DNA, 18 bp, Artificial Sequence
  Description of Artificial Sequence Synthetic primer
cagccatacagcaaatcc

SEQ ID NO: 6 - DNA, 20 bp, Artificial Sequence
  Description of Artificial Sequence Synthetic primer
tcatccgctatgctggctac

SEQ ID NO: 7 - DNA, 23 bp, Artificial Sequence
  Description of Artificial Sequence Synthetic primer
tctcatttcgtgatggagactgc

SEQ ID NO: 8 - DNA, 19 bp, Artificial Sequence
  Description of Artificial Sequence Synthetic primer
ccaacgtgacggacttccc

SEQ ID NO: 9 - DNA, 20 bp, Artificial Sequence
  Description of Artificial Sequence Synthetic primer
tcaaggcgcatgtgaactcc

SEQ ID NO: 10 - DNA, 19 bp, Artificial Sequence
  Description of Artificial Sequence Synthetic primer
ggccagatcctgtccaagc

SEQ ID NO: 11 - DNA, 19 bp, Artificial Sequence
  Description of Artificial Sequence Synthetic primer
gggcagagttggatacccc

SEQ ID NO: 12 - DNA, 23 bp, Artificial Sequence
  Description of Artificial Sequence Synthetic primer
ttttgatgcgtgtctcattacca

SEQ ID NO: 13 - DNA, 21 bp, Artificial Sequence
  Description of Artificial Sequence Synthetic primer
ggacaagcagtgaccatcaag

SEQ ID NO: 14 - DNA, 21 bp, Artificial Sequence
  Description of Artificial Sequence Synthetic primer
gtggaaacttgcatggacaac

SEQ ID NO: 15 - DNA, 20 bp, Artificial Sequence
  Description of Artificial Sequence Synthetic primer
tcgccagcaacctgaatctc

SEQ ID NO: 16 - DNA, 21 bp, Artificial Sequence
  Description of Artificial Sequence Synthetic primer
gtgaagcccaatgcaaacaga

SEQ ID NO: 17 - DNA, 22 bp, Artificial Sequence
  Description of Artificial Sequence Synthetic primer
ggggtcattgatggcaacaata

SEQ ID NO: 18 - DNA, 20 bp, Artificial Sequence
  Description of Artificial Sequence Synthetic primer
tggcctcatagtcaaagaca

SEQ ID NO: 19 - DNA, 18 bp, Artificial Sequence
  Description of Artificial Sequence Synthetic primer
ggctccaggtgaagcagc

SEQ ID NO: 20 - DNA, 21 bp, Artificial Sequence
  Description of Artificial Sequence Synthetic primer
acctctggattgcttgtgaaa

SEQ ID NO: 21 - DNA, 18 bp, Artificial Sequence
  Description of Artificial Sequence Synthetic primer
atcctgtccgggtacaat

SEQ ID NO: 22 - DNA, 21 bp, Artificial Sequence
  Description of Artificial Sequence Synthetic primer
cccgaaaccactcgtatttgg

SEQ ID NO: 23 - DNA, 21 bp, Artificial Sequence
  Description of Artificial Sequence Synthetic primer
gtgtcccgttcttgcatttgc

SEQ ID NO: 24 - DNA, 21 bp, Artificial Sequence
  Description of Artificial Sequence Synthetic primer
tacacgactatgcggtacagc

SEQ ID NO: 25 - DNA, 22 bp, Artificial Sequence
  Description of Artificial Sequence Synthetic primer
gatgtcaaactcactcatggct

SEQ ID NO: 26 - DNA, 21 bp, Artificial Sequence
  Description of Artificial Sequence Synthetic primer
gtgggtttccaccattagcac

SEQ ID NO: 27 - DNA, 21 bp, Artificial Sequence
  Description of Artificial Sequence Synthetic primer
tgcgtgtgggttgtagcaata

SEQ ID NO: 28 - DNA, 21 bp, Artificial Sequence
  Description of Artificial Sequence Synthetic primer
attttccctcaagcgggttgt

SEQ ID NO: 29 - DNA, 23 bp, Artificial Sequence
  Description of Artificial Sequence Synthetic primer
cccagaattaccaagtgagtcct

SEQ ID NO: 30 - DNA, 21 bp, Artificial Sequence
  Description of Artificial Sequence Synthetic primer
aatcctggcacatcgggaatc

SEQ ID NO: 31 - DNA, 20 bp, Artificial Sequence
  Description of Artificial Sequence Synthetic primer
gcacgaagctcttagcgtca

SEQ ID NO: 32 - DNA, 21 bp, Artificial Sequence
  Description of Artificial Sequence Synthetic primer
agcgtgggttaaagtggaagg